SEQUENCE LISTING (41 sequences)

SEQ ID NO: 1 - protein, 447 aa, synthetic construct
  source 1..447: note = Heavy Chain Amino Acid Sequence; Anti-LAG-3 mAb (BMS-986016)
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 214 aa, synthetic construct
  source 1..214: note = Light Chain Amino Acid Sequence; Anti-LAG-3 mAb (BMS-986016)
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 120 aa, synthetic construct
  source 1..120: note = Heavy Chain Variable Region (VH) Amino Acid Sequence; Anti-LAG-3 mAb (BMS-986016)
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 4 - DNA, 360 bp, synthetic construct
  source 1..360: mol_type = other DNA; note = Heavy Chain Variable Region (VH) Nucleotide Sequence; Anti-LAG-3 mAb (BMS-986016)
caggtgcagctacagcagtggggcgcaggactgttgaagccttcggagaccctgtccctcacctgcgctgtctatggtgggtccttcagtgattactactggaactggatccgccagcccccagggaaggggctggagtggattggggaaatcaatcatcgtggaagcaccaactccaacccgtccctcaagagtcgagtcaccctatcactagacacgtccaagaaccagttctccctgaagctgaggtctgtgaccgccgcggacacggctgtgtattactgtgcgtttggatatagtgactacgagtacaactggttcgacccctggggccagggaaccctggtcaccgtctcctca

SEQ ID NO: 5 - protein, 107 aa, synthetic construct
  source 1..107: note = Light Chain Variable Region (VL) Amino Acid Sequence; Anti-LAG-3 mAb (BMS-986016)
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 6 - DNA, 321 bp, synthetic construct
  source 1..321: mol_type = other DNA; note = Light Chain Variable Region (VL) Nucleotide Sequence; Anti-LAG-3 mAb (BMS-986016)
gaaattgtgttgacacagtctccagccaccctgtctttgtctccaggggaaagagccaccctctcctgcagggccagtcagagtattagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcacttttggccaggggaccaacctggagatcaaa

SEQ ID NO: 7 - protein, 5 aa, synthetic construct
  source 1..5: note = Heavy Chain CDR1 Amino Acid Sequence; Anti-LAG-3 mAb (BMS-986016)
DYYWN

SEQ ID NO: 8 - protein, 16 aa, synthetic construct
  source 1..16: note = Heavy Chain CDR2 Amino Acid Sequence; Anti-LAG-3 mAb (BMS-986016)
EINHRGSTNSNPSLKS

SEQ ID NO: 9 - protein, 12 aa, synthetic construct
  source 1..12: note = Heavy Chain CDR3 Amino Acid Sequence; Anti-LAG-3 mAb (BMS-986016)
GYSDYEYNWFDP

SEQ ID NO: 10 - protein, 11 aa, synthetic construct
  source 1..11: note = Light Chain CDR1 Amino Acid Sequence; Anti-LAG-3 mAb (BMS-986016)
RASQSISSYLA

SEQ ID NO: 11 - protein, 7 aa, synthetic construct
  source 1..7: note = Light Chain CDR2 Amino Acid Sequence; Anti-LAG-3 mAb (BMS-986016)
DASNRAT

SEQ ID NO: 12 - protein, 9 aa, synthetic construct
  source 1..9: note = Light Chain CDR3 Amino Acid Sequence; Anti-LAG-3 mAb (BMS-986016)
QQRSNWPLT

SEQ ID NO: 13 - protein, 440 aa, synthetic construct
  source 1..440: note = Heavy Chain Amino Acid Sequence; Anti-PD-1 mAb (BMS936558)
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 213 aa, synthetic construct
  source 1..213: note = Light Chain Amino Acid Sequence; Anti-PD-1 mAb (BMS936558)
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLLSKADYEKHKVYACEVTHQGLSSPVTKSFNRGEC

SEQ ID NO: 15 - protein, 113 aa, synthetic construct [sequence omitted]

SEQ ID NO: 16 - DNA, 339 bp, synthetic construct
  source 1..339: mol_type = other DNA; note = Heavy Chain Variable Region (VH) Nucleotide Sequence; Anti-PD-1 mAb (BMS936558)
caggtgcagctggtggagtctgggggaggcgtggtccagcctgggaggtccctgagactcgactgtaaagcgtctggaatcaccttcagtaactctggcatgcactgggtccgccaggctccaggcaaggggctggagtgggtggcagttatttggtatgatggaagtaaaagatactatgcagactccgtgaagggccgattcaccatctccagagacaattccaagaacacgctgtttctgcaaatgaacagcctgagagccgaggacacggctgtgtattactgtgcgacaaacgacgactactggggccagggaaccctggtcaccgtctcctca

SEQ ID NO: 17 - protein, 107 aa, synthetic construct [sequence omitted]

SEQ ID NO: 18 - DNA, 321 bp, synthetic construct
  source 1..321: mol_type = other DNA; note = Light Chain Variable Region (VL) Nucleotide Sequence; Anti-PD-1 mAb (BMS936558)
gaaattgtgttgacacagtctccagccaccctgtctttgtctccaggggaaagagccaccctctcctgcagggccagtcagagtgttagtagttacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagagtagcaactggcctcggacgttcggccaagggaccaaggtggaaatcaaa

SEQ ID NO: 19 - protein, 5 aa, synthetic construct
  source 1..5: note = Heavy Chain CDR1 Amino Acid Sequence; Anti-PD-1 mAb (BMS936558)
NSGMH

SEQ ID NO: 20 - protein, 17 aa, synthetic construct
  source 1..17: note = Heavy Chain CDR2 Amino Acid Sequence; Anti-PD-1 mAb (BMS936558)
VIWYDGSKRYYADSVKG

SEQ ID NO: 21 - protein, 4 aa, synthetic construct
  source 1..4: note = Heavy Chain CDR3 Amino Acid Sequence; Anti-PD-1 mAb (BMS936558)
NDDY

SEQ ID NO: 22 - protein, 11 aa, synthetic construct
  source 1..11: note = Light Chain CDR1 Amino Acid Sequence; Anti-PD-1 mAb (BMS936558)
RASQSVSSYLA

SEQ ID NO: 23 - protein, 7 aa, synthetic construct
  source 1..7: note = Light Chain CDR2 Amino Acid Sequence; Anti-PD-1 mAb (BMS936558)
DASNRAT

SEQ ID NO: 24 - protein, 9 aa, synthetic construct
  source 1..9: note = Light Chain CDR3 Amino Acid Sequence; Anti-PD-1 mAb (BMS936558)
QQSSNWPRT

SEQ ID NO: 25 - DNA, 1344 bp, synthetic construct
  source 1..1344: mol_type = other DNA; note = Heavy Chain Nucleotide Sequence; Anti-LAG-3 mAb (BMS-986016)
caggtgcagctacagcagtggggcgcaggactgttgaagccttcggagaccctgtccctcacctgcgctgtctatggtgggtccttcagtgattactactggaactggatccgccagcccccagggaaggggctggagtggattggggaaatcaatcatcgtggaagcaccaactccaacccgtccctcaagagtcgagtcaccctatcactagacacgtccaagaaccagttctccctgaagctgaggtctgtgaccgccgcggacacggctgtgtattactgtgcgtttggatatagtgactacgagtacaactggttcgacccctggggccagggaaccctggtcaccgtctcctcagctagcaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

SEQ ID NO: 26 - DNA, 645 bp, synthetic construct
  source 1..645: mol_type = other DNA; note = Light Chain Nucleotide Sequence; Anti-LAG-3 mAb (BMS-986016)
gaaattgtgttgacacagtctccagccaccctgtctttgtctccaggggaaagagccaccctctcctgcagggccagtcagagtattagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctctcacttttggccaggggaccaacctggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 27 - protein, 8 aa, synthetic construct
  source 1..8: note = LAG-3 epitope
PGHPLAPG

SEQ ID NO: 28 - protein, 8 aa, synthetic construct
  source 1..8: note = LAG-3 epitope
HPAAPSSW

SEQ ID NO: 29 - protein, 8 aa, synthetic construct
  source 1..8: note = LAG-3 epitope
PAAPSSWG

SEQ ID NO: 30 - protein, 446 aa, synthetic construct
  source 1..446: note = Heavy Chain Amino Acid Sequence; Anti-LAG-3 mAb (BMS-986016) without terminal lysine
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - DNA, 325 bp, synthetic construct
  source 1..325: mol_type = other DNA; note = light chain variable region (Vk)
gaaattgtgttgacgcagtctccaggcaccctgtctttgtctccaggggaaagagccaccctctcctgcagggccagtcagagtgttggcagcagctacttagcctggtaccagcagaaacctggccaggctcccaggctcctcatctatggtgcattcagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaac

SEQ ID NO: 32 - protein, 108 aa, synthetic construct
  source 1..108: note = light chain variable region predicted sequence
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 33 - DNA, 355 bp, synthetic construct
  source 1..355: mol_type = other DNA; note = heavy chain variable region
caggtgcagctggtggagtctgggggaggcgtggtccagcctgggaggtccctgagactctcctgtgcagcctctggattcaccttcagtagctatactatgcactgggtccgccaggctccaggcaaggggctggagtgggtgacatttatatcatatgatggaaacaataaatactacgcagactccgtgaagggccgattcaccatctccagagacaattccaagaacacgctgtatctgcaaatgaacagcctgagagctgaggacacggctatatattactgtgcgaggaccggctggctggggccctttgactactggggccagggaaccctggtcaccgtctcctcag

SEQ ID NO: 34 - protein, 118 aa, synthetic construct
  source 1..118: note = heavy chain variable region predicted sequence
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 35 - protein, 5 aa, synthetic construct
  source 1..5: note = Heavy Chain CDR1
SYTMH

SEQ ID NO: 36 - protein, 17 aa, synthetic construct
  source 1..17: note = Heavy Chain CDR2
FISYDGNNKYYADSVKG

SEQ ID NO: 37 - protein, 9 aa, synthetic construct
  source 1..9: note = Heavy Chain CDR3
TGWLGPFDY

SEQ ID NO: 38 - protein, 12 aa, synthetic construct
  source 1..12: note = Light Chain CDR1
RASQSVGSSYLA

SEQ ID NO: 39 - protein, 7 aa, synthetic construct
  source 1..7: note = Light Chain CDR2
GAFSRAT

SEQ ID NO: 40 - protein, 9 aa, synthetic construct
  source 1..9: note = Light Chain CDR3
QQYGSSPWT

SEQ ID NO: 41 - protein, 525 aa, synthetic construct
  source 1..525: note = Lymphocyte Activation Gene 3 Protein Amino Acid Sequence (Homo Sapiens, NP_002277)
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

(19) United States
(12) Patent Application Publication
(10) Pub. No.: US 2026/0109766 A1
(43) Pub. Date: Apr. 23, 2026

(54) LAG-3 ANTAGONIST THERAPY FOR MELANOMA

(71) Applicant: Bristol-Myers Squibb Company, Princeton, NJ (US)

(72) Inventors: Shivani SRIVASTAVA, Princeton, NJ (US); Mena ABASKHAROUN, Princeton, NJ (US)

(21) Appl. No.: 19/327,078

(22) Filed: Sep. 12, 2025

Related U.S. Application Data
(63) Continuation of application No. 17/775,067, now abandoned.
(60) Provisional application No. 62/932,916, filed on Nov. 8, 2019.

(51) Int. Cl. C07K 16/28; A61K 39/00; A61K 39/395; A61P 17/00; A61P 35/00; A61P 35/04

(52) U.S. Cl. CPC C07K 16/2803; A61K 39/3955; A61P 17/00; A61P 35/00; A61P 35/04; C07K 16/2818; C07K 16/2827; A61K 2039/507; A61K 2039/54; A61K 2039/545; C07K 2317/21; C07K 2317/565; C07K 2317/90; C07K 2317/94; C07K 2319/00

(57) ABSTRACT

The disclosure provides a method of treating unresectable or metastatic melanoma in a human patient with a lymphocyte activation gene-3 (LAG-3) antagonist. In some aspects, the method includes a combination of the LAG-3 antagonist with a cytotoxic T-lymphocyte antigen-4 (CTLA-4) inhibitor. In some aspects, the method includes one or more additional therapeutic agents and/or anti-cancer therapies.

### CROSS REFERENCE TO RELATED APPLICATIONS

This application is a continuation of U.S. application Ser. No. 17/775,067, which is the National Stage of International Application No. PCT/US2020/059411, filed Nov. 6, 2020, which claims the priority benefit of U.S. Provisional Application No. 62/932,916, filed Nov. 8, 2019, which are incorporated herein by reference in their entireties.

### REFERENCE TO SEQUENCE LISTING SUBMITTED ELECTRONICALLY

The content of the electronically submitted sequence listing (Name: 3338_2040002_SequenceListing_ST26.xml; Size: 52,378 Bytes; and Date of Creation: Dec. 22, 2025) is herein incorporated by reference in its entirety.

### FIELD OF THE INVENTION

The invention disclosed herein relates to methods of treating unresectable or metastatic melanoma in a human patient comprising a lymphocyte activation gene-3 (LAG-3) antagonist.

### BACKGROUND OF THE INVENTION

Human cancers harbor numerous genetic and epigenetic alterations, generating neoantigens potentially recognizable by the immune system (Sjoblom et al. (2006) Science 314:268-74). The adaptive immune system, comprised of T and B lymphocytes, has powerful anti-cancer potential, with a broad capacity and exquisite specificity to respond to diverse tumor antigens. Further, the immune system demonstrates considerable plasticity and a memory component. The successful harnessing of all these attributes of the adaptive immune system would make immunotherapy unique among all cancer treatment modalities.

Lymphocyte activation gene-3 (LAG-3; CD223) is a type I transmembrane protein that is expressed on the cell surface of activated CD4+ and CD8+ T cells and subsets of NK and dendritic cells (Triebel F, et al., J. Exp. Med. 1990; 171:1393-1405; Workman C J, et al., J. Immunol. 2009; 182(4):1885-91). LAG-3 is closely related to CD4, which is a co-receptor for T helper cell activation. Both molecules have 4 extracellular Ig-like domains and bind to major histocompatibility complex (MHC) class II. In contrast to CD4, LAG-3 is only expressed on the cell surface of activated T cells and its cleavage from the cell surface terminates LAG-3 signaling. LAG-3 can also be found as a soluble protein but its function is unknown.

Cytotoxic T-lymphocyte antigen-4 (CTLA-4) is an immunoinhibitory receptor belonging to the CD28 family. CTLA-4 is expressed exclusively on T cells in vivo, and binds to two ligands, CD80 and CD86 (also called B7-1 and B7-2, respectively).

It is an object of the present invention to provide improved methods for treating unresectable or metastatic melanoma.

### SUMMARY OF THE INVENTION

The present disclosure is directed to a method of treating unresectable or metastatic melanoma in a human patient, the method comprising administering to the patient: (a) a lymphocyte activation gene-3 (LAG-3) antagonist, and (b) a cytotoxic T-lymphocyte antigen-4 (CTLA-4) inhibitor; wherein the patient has a sensitizing mutation for a targeted inhibitor therapy.

The present disclosure is directed to a method of treating unresectable or metastatic melanoma in a human patient, the method comprising administering to the patient: (a) a LAG-3 antagonist, and (b) a CTLA-4 inhibitor; wherein the patient has received a prior PD-1 pathway inhibitor as a treatment for melanoma.

In some aspects, the method is a first line therapy.

In some aspects, the method is a second line therapy.

In some aspects, the method is a third line therapy.

In some aspects, the patient has progressed on a prior therapy.

In some aspects, the patient has not received a prior systemic therapy for cancer, the patient has not received a prior systemic therapy for melanoma, or the patient has not received a prior systemic therapy for unresectable or metastatic melanoma.

In some aspects, the patient is naïve to prior immuno-oncology therapy, the patient is naïve to prior immuno-oncology therapy for melanoma, or the melanoma is naïve to prior immuno-oncology therapy.

In some aspects, the patient has histologically confirmed unresectable stage III or stage IV melanoma.

In some aspects, the patient has an Eastern Cooperative Oncology Group (ECOG) performance status of 0 or 1.

In some aspects, the patient has a B-rapidly accelerated fibrosarcoma proto-oncogene (BRAF), mitogen-activated extracellular signal-regulated kinase kinase (MEK), neuroblastoma RAS viral oncogene homolog (NRAS), and/or proto-oncogene c-KIT (KIT) mutation sensitive to targeted inhibitor therapy. In some aspects the patient has a BRAF mutation sensitive to targeted inhibitor therapy.

In some aspects, one or more immune cells in tumor tissue from the patient express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells are tumor-infiltrating lymphocytes. In some aspects, the tumor-infiltrating lymphocytes are CD8⁺ cells.

In some aspects, one or more tumor cells in tumor tissue from the patient express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1.

In some aspects, the LAG-3 antagonist is an anti-LAG-3 antibody.

In some aspects, the anti-LAG-3 antibody is a full-length antibody.

In some aspects, the anti-LAG-3 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), IMP731 (H5L7BW), MK4280 (28G-10), REGN3767 (fianlimab), GSK2831781, humanized BAP050, IMP-701 (LAG525, ieramilimab), aLAG3(0414), aLAG3(0416), Sym022, TSR-033, TSR-075, XmAb22841, MGD013, B1754111, FS118, P 13B02-30, AVA-017, 25F7, AGEN1746, or comprises an antigen binding portion thereof.

In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5.

In some aspects, the anti-LAG-3 antibody comprises: (a) a heavy chain variable region CDR1 comprising the sequence set forth in SEQ ID NO:7; (b) a heavy chain variable region CDR2 comprising the sequence set forth in SEQ ID NO: 8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12.

In some aspects, the anti-LAG-3 antibody comprises heavy and light chain variable regions comprising the sequences set forth in SEQ ID NOs:3 and 5, respectively.

In some aspects, the anti-LAG-3 antibody comprises heavy and light chains comprising the sequences set forth in SEQ ID NOs:1 and 2, respectively.

In some aspects, the anti-LAG-3 antibody comprises heavy and light chains comprising the sequences set forth in SEQ ID NOs:30 and 2, respectively.

In some aspects, the LAG-3 antagonist is a soluble LAG-3 polypeptide. In some aspects, the soluble LAG-3 polypeptide is a fusion polypeptide. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO:41. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha).

In some aspects, the CTLA-4 inhibitor is an anti-CTLA-4 antibody.

In some aspects, the anti-CTLA-4 antibody is a full-length antibody.

In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK-1308, AGEN-1884, or comprises an antigen binding portion thereof.

In some aspects, the anti-CTLA-4 antibody comprises CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

In some aspects, the anti-CTLA-4 antibody comprises: (a) a heavy chain variable region CDR1 comprising the sequence set forth in SEQ ID NO:35; (b) a heavy chain variable region CDR2 comprising the sequence set forth in SEQ ID NO:36; (c) a heavy chain variable region CDR3 comprising the sequence set forth in SEQ ID NO:37; (d) a light chain variable region CDR1 comprising the sequence set forth in SEQ ID NO:38; (e) a light chain variable region CDR2 comprising the sequence set forth in SEQ ID NO:39; and (f) a light chain variable region CDR3 comprising the sequence set forth in SEQ ID NO:40.

In some aspects, the anti-CTLA-4 antibody comprises heavy and light chain variable regions comprising the sequences set forth in SEQ ID NOs:34 and 32, respectively.

In some aspects, the anti-CTLA-4 antibody comprises the heavy and light chains of ipilimumab.

In some aspects, the LAG-3 antagonist and the CTLA-4 inhibitor are formulated for intravenous administration.

In some aspects, the LAG-3 antagonist and the CTLA-4 inhibitor are formulated separately.

In some aspects, the LAG-3 antagonist and the CTLA-4 inhibitor are formulated together.

In some aspects, the LAG-3 antagonist is administered before the CTLA-4 inhibitor.

In some aspects, the CTLA-4 inhibitor is administered before the LAG-3 antagonist.

In some aspects, the LAG-3 antagonist and the CTLA-4 inhibitor are administered concurrently.

In some aspects, the LAG-3 antagonist and/or the CTLA-4 inhibitor is administered at a flat dose.

In some aspects, the LAG-3 antagonist and/or the CTLA-4 inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG-3 antagonist and/or the CTLA-4 inhibitor is administered at a dose of about 0.25 mg, about 0.5 mg, about 0.75 mg, about 1 mg, about 1.25 mg, about 1.5 mg, about 1.75 mg, about 2 mg, 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist and/or the CTLA-4 inhibitor is administered at a weight-based dose.

In some aspects, the LAG-3 antagonist and/or the CTLA-4 inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG-3 antagonist and/or the CTLA-4 inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the LAG-3 antagonist and/or the CTLA-4 inhibitor is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the method further comprises administering to the patient an additional therapeutic agent. In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor comprises dabrafenib, vemurafenib, encorafenib, trametinib, cobimetinib, binimetinib, or any combination thereof.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGF receptor (EGFR), or any combination thereof.

In some aspects, the anti-angiogenesis agent comprises bevacizumab, ramucirumab, aflibercept, tanibirumab, olaratumab, nesvacumab, AMG780, MEDI3617, vanucizumab, rilotumumab, ficlatuzumab, TAK-701, onartuzumab, emibetuzumab, or any combination thereof.

In some aspects, the checkpoint inhibitor comprises a programmed death-1 (PD-1) pathway inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-0) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor comprises a PD-1 pathway inhibitor.

In some aspects, the PD-1 pathway inhibitor is an anti-PD-1 antibody and/or an anti-PD-L1 antibody.

In some aspects, the PD-1 pathway inhibitor is an anti-PD-1 antibody.

In some aspects, the anti-PD-1 antibody is a full-length antibody.

In some aspects, the anti-PD-1 antibody is a monoclonal, human, humanized, chimeric, or multispecific antibody. In some aspects, the multispecific antibody is a DART, a DVD-Ig, or bispecific antibody.

In some aspects, the anti-PD-1 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-PD-1 antibody is nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224.

In some aspects, the PD-1 pathway inhibitor is an anti-PD-L1 antibody.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, CK-301, or comprises an antigen binding portion thereof.

In some aspects, the PD-1 pathway inhibitor is BMS-986189.

The present disclosure is directed to a method of treating unresectable or metastatic melanoma in a human patient, the method comprising administering to the patient: (a) a dose of about 360 mg of an anti-LAG-3 antibody, and (b) a dose of about 3 mg/kg of an anti-CTLA-4 antibody.

The present disclosure is directed to a method of treating unresectable or metastatic melanoma in a human patient, the method comprising administering to the patient: (a) a dose of about 720 mg of an anti-LAG-3 antibody, and (b) a dose of about 3 mg/kg of an anti-CTLA-4 antibody.

The present disclosure is directed to a method of treating unresectable or metastatic melanoma in a human patient, the method comprising administering to the patient: (a) a dose of about 1080 mg of an anti-LAG-3 antibody, and (b) a dose of about 3 mg/kg of an anti-CTLA-4 antibody.

The present disclosure is directed to a method of treating unresectable or metastatic melanoma in a human patient, the method comprising administering to the patient: (a) a dose of about 1200 mg of an anti-LAG-3 antibody, and (b) a dose of about 3 mg/kg of an anti-CTLA-4 antibody.

The present disclosure is directed to a method of treating unresectable or metastatic melanoma in a human patient, the method comprising administering to the patient: (a) a dose of about 360 mg of an anti-LAG-3 antibody, and (b) a dose of about 3 mg/kg of an anti-CTLA-4 antibody; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 720 mg of an anti-LAG-3 antibody, and (b) a dose of about 3 mg/kg of an anti-CTLA-4 antibody; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 1080 mg of an anti-LAG-3 antibody, and (b) a dose of about 3 mg/kg of an anti-CTLA-4 antibody; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 1200 mg of an anti-LAG-3 antibody, and (b) a dose of about 3 mg/kg of an anti-CTLA-4 antibody; wherein the patient has received a prior PD-1 pathway inhibitor as a treatment for melanoma.

In some aspects, the anti-LAG-3 antibody and the anti-CTLA-4 antibody are administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the patient has histologically confirmed unresectable stage III or stage IV melanoma.

In some aspects, the patient has an Eastern Cooperative Oncology Group (ECOG) performance status of 0 or 1.

In some aspects, one or more immune cells in tumor tissue from the patient express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells are tumor-infiltrating lymphocytes. In some aspects, the tumor-infiltrating lymphocytes are CD8⁺ cells. In some aspects, greater than about 1% of the patient's tumor infiltrating lymphocytes cells express LAG-3.

In some aspects, one or more tumor cells in tumor tissue from the patient express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1. In some aspects, greater than about 1% of the patient's tumor cells express PD-L1.

In some aspects, the patient's tumor cells contain a BRAF V600 mutation.

In some aspects, the anti-LAG-3 antibody and/or the anti-CTLA-4 antibody is a full-length antibody.

In some aspects, the anti-LAG-3 antibody and/or the anti-CTLA-4 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG-3 antibody and/or the anti-CTLA-4 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), IMP731 (H5L7BW), MK4280 (28G-10), REGN3767 (fianlimab), GSK2831781, humanized BAP050, IMP-701 (LAG-525, ieramilimab), aLAG3(0414), aLAG3(0416), Sym022, TSR-033, TSR-075, XmAb22841, MGD013, BI754111, FS118, P 13B02-30, AVA-017, 25F7, AGEN1746, or comprises an antigen binding portion thereof.

In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5.

In some aspects, the anti-LAG-3 antibody comprises: (a) a heavy chain variable region CDR1 comprising the sequence set forth in SEQ ID NO:7; (b) a heavy chain variable region CDR2 comprising the sequence set forth in SEQ ID NO: 8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12.

In some aspects, the anti-LAG-3 antibody comprises heavy and light chain variable regions comprising the sequences set forth in SEQ ID NOs:3 and 5, respectively.

In some aspects, the anti-LAG-3 antibody comprises heavy and light chains comprising the sequences set forth in SEQ ID NOs:1 and 2, respectively.

In some aspects, the anti-LAG-3 antibody comprises heavy and light chains comprising the sequences set forth in SEQ ID NOs:30 and 2, respectively.

In some aspects, the anti-CTLA-4 antibody is ipilimumab, tremelimumab, MK-1308, AGEN-1884, or comprises an antigen binding portion thereof.

In some aspects, the anti-CTLA-4 antibody comprises CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

In some aspects, the anti-CTLA-4 antibody comprises: (a) a heavy chain variable region CDR1 comprising the sequence set forth in SEQ ID NO:35; (b) a heavy chain variable region CDR2 comprising the sequence set forth in SEQ ID NO:36; (c) a heavy chain variable region CDR3 comprising the sequence set forth in SEQ ID NO:37; (d) a light chain variable region CDR1 comprising the sequence set forth in SEQ ID NO:38; (e) a light chain variable region CDR2 comprising the sequence set forth in SEQ ID NO:39; and (f) a light chain variable region CDR3 comprising the sequence set forth in SEQ ID NO:40.

In some aspects, the anti-CTLA-4 antibody comprises heavy and light chain variable regions comprising the sequences set forth in SEQ ID NOs:34 and 32, respectively.

In some aspects, the anti-CTLA-4 antibody comprises the heavy and light chains of ipilimumab.

In some aspects, the method further comprises administering a PD-1 pathway inhibitor. In some aspects, the PD-1 pathway inhibitor is an anti-PD-1 antibody and/or an anti-PD-L1 antibody. In some aspects, the anti-PD-1 antibody is nivolumab, pembrolizumab, PDR001 (spartalizumab), MEDI-0680, TSR-042, REGN2810 (cemiplimab), JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the anti-LAG-3 antibody and anti-CTLA-4 antibody are formulated for intravenous administration.

In some aspects, the anti-LAG-3 antibody and anti-CTLA-4 antibody are formulated separately.

In some aspects, the anti-LAG-3 antibody and anti-CTLA-4 antibody are formulated together.

In some aspects, the anti-LAG-3 antibody and anti-CTLA-4 antibody are administered together.

In some aspects, the anti-LAG-3 antibody and anti-CTLA-4 antibody are administered separately.

In some aspects, the anti-LAG-3 antibody is administered concurrently with the anti-CTLA-4 antibody.

In some aspects, the anti-LAG-3 antibody is administered prior to the administration of the anti-CTLA-4 antibody.

In some aspects, the anti-LAG-3 antibody is administered after the administration of the anti-CTLA-4 antibody.

The present disclosure is directed to a method of treating unresectable or metastatic melanoma in a human patient, the method comprising administering to the patient: (a) an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

The present disclosure is directed to a method of treating unresectable or metastatic melanoma in a human patient, the method comprising administering to the patient: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

The present disclosure is directed to a method of treating unresectable or metastatic melanoma in a human patient, the method comprising administering to the patient: (a) a dose of about 1080 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

The present disclosure is directed to a method of treating unresectable or metastatic melanoma in a human patient, the method comprising administering to the patient: (a) a dose of about 1200 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

The present disclosure is directed to a method of treating unresectable or metastatic melanoma in a human patient, the method comprising administering to the patient: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 1080 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

The present disclosure is directed to a method of treating unresectable or metastatic melanoma in a human patient, the method comprising administering to the patient: (a) a dose of about 1200 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

In some aspects, the anti-LAG-3 antibody and the anti-CTLA-4 antibody are administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the anti-LAG-3 antibody and the anti-CTLA-4 antibody are administered once about every three weeks.

In some aspects, the patient has histologically confirmed unresectable stage III or stage IV melanoma.

In some aspects, the patient has an Eastern Cooperative Oncology Group (ECOG) performance status of 0 or 1.

In some aspects, one or more immune cells in tumor tissue from the patient express LAG-3. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, the immune cells are tumor-infiltrating lymphocytes. In some aspects, the tumor-infiltrating lymphocytes are CD8⁺ cells. In some aspects, greater than about 1% of the patient's tumor infiltrating lymphocytes cells express LAG-3.

In some aspects, one or more tumor cells in tumor tissue from the patient express PD-L1. In some aspects, at least about 1%, at least about 3%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1. In some aspects, greater than 1% of the patient's tumor cells express PD-L1.

In some aspects, the patient's tumor cells contain a BRAF V600 mutation.

The present disclosure is directed to a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising: (i) a heavy chain variable region CDR1 comprising the sequence set forth in SEQ ID NO:7; (ii) a heavy chain variable region CDR2 comprising the sequence set forth in SEQ ID NO:8; (iii) a heavy chain variable region CDR3 comprising the sequence set forth in SEQ ID NO:9; (iv) a light chain variable region CDR1 comprising the sequence set forth in SEQ ID NO:10; (v) a light chain variable region CDR2 comprising the sequence set forth in SEQ ID NO:11; and (vi) a light chain variable region CDR3 comprising the sequence set forth in SEQ ID NO:12, and (b) a dose of about 3 mg/kg of an anti-CTLA-4 antibody comprising: (i) a heavy chain variable region CDR1 comprising the sequence set forth in SEQ ID NO:35; (ii) a heavy chain variable region CDR2 comprising the sequence set forth in SEQ ID NO:36; (iii) a heavy chain variable region CDR3 comprising the sequence set forth in SEQ ID NO:37; (iv) a light chain variable region CDR1 comprising the sequence set forth in SEQ ID NO:38; (v) a light chain variable region CDR2 comprising the sequence set forth in SEQ ID NO:39; and (vi) a light chain variable region CDR3 comprising the sequence set forth in SEQ ID NO:40.

The present disclosure is directed to a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising: (i) a heavy chain variable region CDR1 comprising the sequence set forth in SEQ ID NO:7; (ii) a heavy chain variable region CDR2 comprising the sequence set forth in SEQ ID NO:8; (iii) a heavy chain variable region CDR3 comprising the sequence set forth in SEQ ID NO:9; (iv) a light chain variable region CDR1 comprising the sequence set forth in SEQ ID NO:10; (v) a light chain variable region CDR2 comprising the sequence set forth in SEQ ID NO:11; and (vi) a light chain variable region CDR3 comprising the sequence set forth in SEQ ID NO:12, and (b) a dose of about 3 mg/kg of an anti-CTLA-4 antibody comprising: (i) a heavy chain variable region CDR1 comprising the sequence set forth in SEQ ID NO:35; (ii) a heavy chain variable region CDR2 comprising the sequence set forth in SEQ ID NO:36; (iii) a heavy chain variable region CDR3 comprising the sequence set forth in SEQ ID NO:37; (iv) a light chain variable region CDR1 comprising the sequence set forth in SEQ ID NO:38; (v) a light chain variable region CDR2 comprising the sequence set forth in SEQ ID NO:39; and (vi) a light chain variable region CDR3 comprising the sequence set forth in SEQ ID NO:40; wherein the patient has received a prior PD-1 pathway inhibitor as a treatment for melanoma.

The present disclosure is directed to a method of treating unresectable or metastatic melanoma in a human patient, the method comprising administering to the patient an effective amount of each of: (a) a dose of about 360 mg, about 720 mg, about 1080 mg, or about 1200 mg of an anti-LAG-3 antibody comprising heavy and light chain variable regions comprising the sequences set forth in SEQ ID NOs:3 and 5, respectively, and (b) a dose of about 3 mg/kg of an anti-CTLA-4 antibody comprising heavy and light chain variable regions comprising the sequences set forth in SEQ ID NOs:34 and 32, respectively.

The present disclosure is directed to a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising heavy and light chain variable regions comprising the sequences set forth in SEQ ID NOs:3 and 5, respectively, and (b) a dose of about 3 mg/kg of an anti-CTLA-4 antibody comprising heavy and light chain variable regions comprising the sequences set forth in SEQ ID NOs:34 and 32, respectively; wherein the patient has received a prior PD-1 pathway inhibitor as a treatment for melanoma.

In some aspects, the anti-LAG-3 antibody and the anti-CTLA-4 antibody are administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the anti-LAG-3 antibody and the anti-CTLA-4 antibody are administered once about every three weeks.

In some aspects, the anti-LAG-3 antibody is relatlimab.

In some aspects, the anti-CTLA-4 antibody is ipilimumab.

The present disclosure is directed to a method of treating unresectable or metastatic melanoma in a human patient, the method comprising administering to the patient an anti-LAG-3 antibody comprising: (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12, and wherein the patient has received a prior PD-1 pathway inhibitor as a treatment for melanoma; and wherein at least one dose of the anti-LAG-3 antibody is administered at a dose of about 360 mg, about 720 mg, about 1080 mg, or about 1200 mg.

In some aspects, the patient is further administered chemotherapy.

In some aspects, the patient's tumor cells express fibrinogen-like protein 1 (FGL1).

In some aspects, the presence of BRAF V600E mutation in a tumor specimen is confirmed prior to initiation of treatment. In some aspects, the presence of the BRAF V600E mutation is confirmed using the Cobas® 4800 BRAF V600 Mutation test.

In some aspects, the patient is not additionally administered a PD-1 pathway inhibitor.

### DETAILED DESCRIPTION OF THE INVENTION

The present disclosure provides a method of treating unresectable or metastatic melanoma in a human patient comprising administering to the patient a LAG-3 antagonist (e.g., anti-LAG-3 antibody). In some aspects, the patient has a sensitizing mutation for a targeted inhibitor therapy (e.g., a B-rapidly associated fibrosarcoma proto-oncogene (BRAF) mutation). In some aspects, the method is a first, second, or third line therapy. In some aspects, the patient has received a prior programmed death-1 (PD-1) pathway inhibitor (e.g., an anti-PD-1 antibody) as a treatment for melanoma. In some aspects, the patient has histologically confirmed unresectable stage III or stage IV melanoma. The present disclosure is also directed to methods of treating unresectable or metastatic melanoma in a human patient comprising a combination of a LAG-3 antagonist (e.g., an anti-LAG-3 antibody) and a CTLA-4 inhibitor (e.g., an anti-CTLA-4 antibody). In some aspects, the method further comprises administering one or more additional therapeutic agents (e.g., a PD-1 pathway inhibitor such as an anti-PD-1 antibody) and/or anti-cancer therapies (e.g., a chemotherapy) in combination with the LAG-3 antagonist or in combination with the LAG-3 antagonist and the CTLA-4 inhibitor. In some aspects, the method further comprises: determining LAG-3 and/or PD-L1 expression in tumor tissue from the patient and/or administering the LAG-3 antagonist or the combination of the LAG-3 antagonist and the CTLA-4 inhibitor to the patient based on LAG-3 and/or PD-L1 expression in the patient's tumor tissue.

### I. Terms

In order that the present disclosure may be more readily understood, certain terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleotide sequence,” is understood to represent one or more nucleotide sequences. As such, the terms “a” (or “an”), “one or more,” and “at least one” can be used interchangeably herein.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

The terms “about” or “comprising essentially of” refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of” can mean within 1 or more than 1 standard deviation per the practice in the art. Alternatively, “about” or “comprising essentially of” can mean a range of up to 10% or 20% (i.e., ±10% or ±20%). For example, about 3 mg can include any number between 2.7 mg and 3.3 mg (for 10%) or between 2.4 mg and 3.6 mg (for 20%). Furthermore, particularly with respect to biological systems or processes, the terms can mean up to an order of magnitude or up to 5-fold of a value. When particular values or compositions are provided in the application and claims, unless otherwise stated, the meaning of “about” or “comprising essentially of” should be assumed to be within an acceptable error range for that particular value or composition.

As described herein, any concentration range, percentage range, ratio range or integer range is to be understood to include the value of any integer within the recited range and, when appropriate, fractions thereof (such as one-tenth and one-hundredth of an integer), unless otherwise indicated.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 5th ed., 2013, Academic Press; and the Oxford Dictionary Of Biochemistry And Molecular Biology, 2006, Oxford University Press, provide one of skill with a general dictionary of many of the terms used in this disclosure.

Units, prefixes, and symbols are denoted in their Systeme International de Unites (SI) accepted form. Numeric ranges are inclusive of the numbers defining the range.

The headings provided herein are not limitations of the various aspects of the disclosure, which can be had by reference to the specification as a whole. Accordingly, the terms defined immediately below are more fully defined by reference to the specification in its entirety.

An “antagonist” shall include, without limitation, any molecule capable of blocking, reducing, or otherwise limiting an interaction or activity of a target molecule (e.g., LAG-3). In some aspects, the antagonist is an antibody. In other aspects, the antagonist comprises a small molecule. The terms “inhibitor” and “antagonist” are used interchangeably herein.

An “antibody” (Ab) shall include, without limitation, a glycoprotein immunoglobulin which binds specifically to an antigen and comprises at least two heavy (H) chains and two light (L) chains interconnected by disulfide bonds. Each H chain comprises a heavy chain variable region (abbreviated herein as V_{H}) and a heavy chain constant region (abbreviated herein as C_{H}). The heavy chain constant region comprises three constant domains, C_{H1}, C_{H2} and C_{H3}. Each light chain comprises a light chain variable region (abbreviated herein as V_{L}) and a light chain constant region (abbreviated herein as C_{L}). The light chain constant region comprises one constant domain, C_{L}. The V_{H} and V_{L} regions can be further subdivided into regions of hypervariability, termed complementarity determining regions (CDRs), interspersed with regions that are more conserved, termed framework regions (FR). Each V_{H} and V_{L} comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. A heavy chain can have the C-terminal lysine or not. Unless specified otherwise herein, the amino acids in the variable regions are numbered using the Kabat numbering system and those in the constant regions are numbered using the EU system.

An immunoglobulin can derive from any of the commonly known isotypes, including but not limited to IgA, secretory IgA, IgG, and IgM. IgG subclasses are also well known to those in the art and include but are not limited to human IgG1, IgG2, IgG3, and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; single chain antibodies; monospecific antibodies; bispecific antibodies; and multi-specific antibodies. A nonhuman antibody can be humanized by recombinant methods to reduce its immunogenicity in humans. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, that retains the ability to bind specifically to the antigen bound by the whole immunoglobulin. Examples of an “antigen-binding portion” or “antigen-binding fragment” include: (1) a Fab fragment (fragment from papain cleavage) or a similar monovalent fragment consisting of the V_{L}, V_{H}, L_{C} and C_{H1} domains; (2) a F(ab′)2 fragment (fragment from pepsin cleavage) or a similar bivalent fragment comprising two Fab fragments linked by a disulfide bridge at the hinge region; (3) a Fd fragment consisting of the VH and CH1 domains; (4) a Fv fragment consisting of the V_{L} and V_{H} domains of a single arm; (5) a single domain antibody (dAb) fragment (Ward et al., (1989) Nature 341:544-46), which consists of a V_{H} domain; (6) a bi-single domain antibody which consists of two V_{H} domains linked by a hinge (dual-affinity re-targeting antibodies (DARTs)); or (7) a dual variable domain immunoglobulin. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883).

An “isolated antibody” refers to an antibody that is substantially free of other antibodies having different antigenic specificities (e.g., an isolated antibody that binds specifically to LAG-3 is substantially free of antibodies that do not bind specifically to LAG-3). An isolated antibody that binds specifically to LAG-3 can, however, have cross-reactivity to other antigens, such as LAG-3 molecules from different species. Moreover, an isolated antibody can be substantially free of other cellular material and/or chemicals.

The term “monoclonal antibody” (“mAb”) refers to a non-naturally occurring preparation of antibody molecules of single molecular composition, i.e., antibody molecules whose primary sequences are essentially identical, and which exhibits a single binding specificity and affinity for a particular epitope. A mAb is an example of an isolated antibody. MAbs can be produced by hybridoma, recombinant, transgenic or other techniques known to those skilled in the art.

A “human” antibody (HuMAb) refers to an antibody having variable regions in which both the framework and CDR regions are derived from human germline immunoglobulin sequences. Furthermore, if the antibody contains a constant region, the constant region is also derived from human germline immunoglobulin sequences. The human antibodies of the invention can include amino acid residues not encoded by human germline immunoglobulin sequences (e.g., mutations introduced by random or site-specific mutagenesis in vitro or by somatic mutation in vivo). However, the term “human antibody,” as used herein, is not intended to include antibodies in which CDR sequences derived from the germline of another mammalian species, such as a mouse, have been grafted onto human framework sequences. The terms “human” antibodies and “fully human” antibodies and are used synonymously.

A “humanized antibody” refers to an antibody in which some, most or all of the amino acids outside the CDR domains of a non-human antibody are replaced with corresponding amino acids derived from human immunoglobulins. In one aspect of a humanized form of an antibody, some, most or all of the amino acids outside the CDR domains have been replaced with amino acids from human immunoglobulins, whereas some, most or all amino acids within one or more CDR regions are unchanged. Small additions, deletions, insertions, substitutions or modifications of amino acids are permissible as long as they do not abrogate the ability of the antibody to bind to a particular antigen. A “humanized” antibody retains an antigenic specificity similar to that of the original antibody.

A “chimeric antibody” refers to an antibody in which the variable regions are derived from one species and the constant regions are derived from another species, such as an antibody in which the variable regions are derived from a mouse antibody and the constant regions are derived from a human antibody.

An “anti-antigen” antibody refers to an antibody that binds specifically to the antigen. For example, an anti-LAG-3 antibody binds specifically to LAG-3.

“LAG-3” refers to Lymphocyte Activation Gene-3. The term “LAG-3” includes variants, isoforms, homologs, orthologs and paralogs. For example, antibodies specific for a human LAG-3 protein can, in certain cases, cross-react with a LAG-3 protein from a species other than human. In other aspects, the antibodies specific for a human LAG-3 protein can be completely specific for the human LAG-3 protein and not exhibit species or other types of cross-reactivity, or can cross-react with LAG-3 from certain other species, but not all other species (e.g., cross-react with monkey LAG-3 but not mouse LAG-3). The term “human LAG-3” refers to human sequence LAG-3, such as the complete amino acid sequence of human LAG-3 having GenBank Accession No. NP_002277. The term “mouse LAG-3” refers to mouse sequence LAG-3, such as the complete amino acid sequence of mouse LAG-3 having GenBank Accession No. NP_032505. LAG-3 is also known in the art as, for example, CD223. The human LAG-3 sequence can differ from human LAG-3 of GenBank Accession No. NP_002277 by having, e.g., conserved mutations or mutations in non-conserved regions, and the LAG-3 has substantially the same biological function as the human LAG-3 of GenBank Accession No. NP_002277. For example, a biological function of human LAG-3 is having an epitope in the extracellular domain of LAG-3 that is specifically bound by an antibody of the instant disclosure or a biological function of human LAG-3 is binding to MHC Class II molecules.

A particular human LAG-3 sequence will generally be at least about 90% identical in amino acid sequence to human LAG-3 of GenBank Accession No. NP_002277 and contains amino acid residues that identify the amino acid sequence as being human when compared to LAG-3 amino acid sequences of other species (e.g., murine). In certain cases, a human LAG-3 can be at least about 95%, or even at least about 96%, at least about 97%, at least about 98%, at least about 99%, or about 100% identical in amino acid sequence to LAG-3 of GenBank Accession No. NP_002277. In certain aspects, a human LAG-3 sequence will display no more than 10 amino acid differences from the LAG-3 sequence of GenBank Accession No. NP_002277. In certain aspects, the human LAG-3 can display no more than 5, or even no more than 4, 3, 2, or 1 amino acid difference from the LAG-3 sequence of GenBank Accession No. NP_002277.

“Programmed Death-1 (PD-1)” refers to an immunoinhibitory receptor belonging to the CD28 family. PD-1 is expressed predominantly on previously activated T cells in vivo, and binds to two ligands, PD-L1 and PD-L2. The term “PD-1” as used herein includes human PD-1 (hPD-1), variants, isoforms, and species homologs of hPD-1, and analogs having at least one common epitope with hPD-1. The complete hPD-1 sequence can be found under GenBank Accession No. U64863. “PD-1” and “PD-1 receptor” are used interchangeably herein.

“Cytotoxic T-Lymphocyte Antigen-4 (CTLA-4)” refers to an immunoinhibitory receptor belonging to the CD28 family. CTLA-4 is expressed exclusively on T cells in vivo, and binds to two ligands, CD80 and CD86 (also called B7-1 and B7-2, respectively). The term “CTLA-4” as used herein includes human CTLA-4 (hCTLA-4), variants, isoforms, and species homologs of hCTLA-4, and analogs having at least one common epitope with hCTLA-4. The complete hCTLA-4 sequence can be found under GenBank Accession No. AAB59385.

“Programmed Death Ligand-1 (PD-L1)” is one of two cell surface glycoprotein ligands for PD-1 (the other being PD-L2) that downregulate T cell activation and cytokine secretion upon binding to PD-1. The term “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

“Programmed Death Ligand-2 (PD-L2)” as used herein includes human PD-L2 (hPD-L2), variants, isoforms, and species homologs of hPD-L2, and analogs having at least one common epitope with hPD-L2. The complete hPD-L2 sequence can be found under GenBank Accession No. Q9BQ51.

A “patient” as used herein includes any patient who is afflicted with unresectable or metastatic melanoma. The terms “subject” and “patient” are used interchangeably herein.

“Administering” refers to the physical introduction of a therapeutic agent to a subject (e.g., a composition or formulation comprising the therapeutic agent), using any of the various methods and delivery systems known to those skilled in the art. Exemplary routes of administration include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some aspects, the therapeutic agent is administered via a non-parenteral route, in some aspects, orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“Treatment” or “therapy” of a subject refers to any type of intervention or process performed on, or the administration of an active agent to, the subject with the objective of reversing, alleviating, ameliorating, inhibiting, slowing down progression, development, severity or recurrence of a symptom, complication or condition, or biochemical indicia associated with a disease. Response Evaluation Criteria In Solid Tumors (RECIST) is a measure for treatment efficacy and are established rules that define when tumors respond, stabilize, or progress during treatment. RECIST 1.1 is the current guideline to solid tumor measurement and definitions for objective assessment of change in tumor size for use in adult and pediatric cancer clinical trials.

As used herein, “effective treatment” refers to treatment producing a beneficial effect, e.g., amelioration of at least one symptom of a disease or disorder. A beneficial effect can take the form of an improvement over baseline, i.e., an improvement over a measurement or observation made prior to initiation of therapy according to the method. A beneficial effect can also take the form of arresting, slowing, retarding, or stabilizing of a deleterious progression of a marker of solid tumor. Effective treatment can refer to alleviation of at least one symptom of a solid tumor. Such effective treatment can, e.g., reduce patient pain, reduce the size and/or number of lesions, can reduce or prevent metastasis of a tumor, and/or can slow tumor growth.

The term “effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to solid tumors, an effective amount comprises an amount sufficient to cause a tumor to shrink and/or to decrease the growth rate of the tumor (such as to suppress tumor growth) or to delay other unwanted cell proliferation. In some aspects, an effective amount is an amount sufficient to prevent or delay tumor recurrence. An effective amount can be administered in one or more administrations. The effective amount of the drug or composition can: (i) reduce the number of cancer cells; (ii) reduce tumor size; (iii) inhibit, retard, slow to some extent and can stop cancer cell infiltration into peripheral organs; (iv) inhibit (i.e., slow to some extent and can stop tumor metastasis; (v) inhibit tumor growth; (vi) prevent or delay occurrence and/or recurrence of tumor; and/or (vii) relieve to some extent one or more of the symptoms associated with the cancer. In one example, an “effective amount” is the amount of anti-LAG-3 antibody alone or the amount of anti-LAG-3 antibody and the amount an additional therapeutic agent (e.g., anti-CTLA-4 antibody), in combination, clinically proven to affect a significant decrease in cancer or slowing of progression of cancer, such as an advanced solid tumor.

As used herein, the terms “fixed dose,” “flat dose,” and “flat-fixed dose” are used interchangeably and refer to a dose that is administered to a patient without regard for the weight or body surface area (BSA) of the patient. The fixed or flat dose is therefore not provided as a mg/kg dose, but rather as an absolute amount of the agent (e.g., an amount in g or mg).

The use of the term “fixed dose combination” with regard to a composition of the invention means that two or more different inhibitors as described herein (e.g., an anti-LAG-3 antibody and an anti-CTLA-4 antibody) in a single composition are present in the composition in particular (fixed) ratios with each other. In some aspects, the fixed dose is based on the weight (e.g., mg) of the inhibitors. In certain aspects, the fixed dose is based on the concentration (e.g., mg/ml) of the inhibitors. In some aspects, the ratio is at least about 1:1, about 1:2, about 1:3, about 1:4, about 1:5, about 1:6, about 1:7, about 1:8, about 1:9, about 1:10, about 1:15, about 1:20, about 1:30, about 1:40, about 1:50, about 1:60, about 1:70, about 1:80, about 1:90, about 1:100, about 1:120, about 1:140, about 1:160, about 1:180, about 1:200, about 200:1, about 180:1, about 160:1, about 140:1, about 120:1, about 100:1, about 90:1, about 80:1, about 70:1, about 60:1, about 50:1, about 40:1, about 30:1, about 20:1, about 15:1, about 10:1, about 9:1, about 8:1, about 7:1, about 6:1, about 5:1, about 4:1, about 3:1, or about 2:1 mg first inhibitor to mg second inhibitor.

The term “weight based dose” as referred to herein means that a dose that is administered to a patient is calculated based on the weight of the patient.

“Dosing interval,” as used herein, means the amount of time that elapses between multiple doses of a formulation disclosed herein being administered to a subject. Dosing interval can thus be indicated as ranges.

The term “dosing frequency” as used herein refers to the frequency of administering doses of a formulation disclosed herein in a given time. Dosing frequency can be indicated as the number of doses per a given time, e.g., once a week or once in two weeks, etc.

The terms “about once a week,” “once about every week,” “once about every two weeks,” or any other similar dosing interval terms as used herein means approximate number, and “about once a week” or “once about every week” can include every seven days±two days, i.e., every five days to every nine days. The dosing frequency of “once a week” thus can be every five days, every six days, every seven days, every eight days, or every nine days. “Once about every three weeks” can include every 21 days±3 days, i.e., every 25 days to every 31 days. Similar approximations apply, for example, to once about every two weeks, once about every four weeks, once about every five weeks, once about every six weeks, once about every seven weeks, once about every eight weeks, once about every nine weeks, once about every ten weeks, once about every eleven weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “tumor” as used herein refers to any mass of tissue that results from excessive cell growth or proliferation, either benign (non-cancerous) or malignant (cancerous), including pre-cancerous lesions.

The term “biological sample” as used herein refers to biological material isolated from a subject. The biological sample can contain any biological material suitable for analysis, for example, by sequencing nucleic acids in the tumor (or circulating tumor cells) and identifying a genomic alteration in the sequenced nucleic acids. The biological sample can be any suitable biological tissue or fluid such as, for example, tumor tissue, blood, blood plasma, and serum. The biological sample can be a test tissue sample (e.g., a tissue sample comprising tumor cells and tumor-infiltrating inflammatory cells). In one aspect, the sample is a tumor tissue biopsy, e.g., a formalin-fixed, paraffin-embedded (FFPE) tumor tissue or a fresh-frozen tumor tissue or the like. In another aspect, the biological sample is a liquid biopsy that, in some aspects, comprises one or more of blood, serum, plasma, circulating tumor cells, exoRNA, ctDNA, and cfDNA.

By way of example, an “anti-cancer agent” promotes cancer regression in a subject. In preferred aspects, a therapeutically effective amount of the agent promotes cancer regression to the point of eliminating the cancer. “Promoting cancer regression” means that administering an effective amount of the anti-cancer agent, alone or in combination with another agent, results in a reduction in tumor growth or size, necrosis of the tumor, a decrease in severity of at least one disease symptom, an increase in frequency and duration of disease symptom-free periods, or a prevention of impairment or disability due to the disease affliction. In addition, the terms “effective” and “effectiveness” with regard to a treatment includes both pharmacological effectiveness and physiological safety. Pharmacological effectiveness refers to the ability of the agent to promote cancer regression in the patient. Physiological safety refers to the level of toxicity, or other adverse physiological effects at the cellular, organ and/or organism level (adverse effects) resulting from administration of the agent.

By way of example for the treatment of tumors, a therapeutically effective amount of an anti-cancer agent can inhibit cell growth or tumor growth by at least about 20%, at least about 40%, at least about 60%, or at least about 80% relative to untreated subjects. In other aspects of the disclosure, tumor regression can be observed and continue for a period of at least about 20 days, more preferably at least about 40 days, or at least about 60 days. Notwithstanding these measurements of therapeutic effectiveness, evaluation of immunotherapeutic drugs must also make allowance for immune-related response patterns.

As used herein, an “immuno-oncology” therapy or an “I-O” or “IO” therapy refers to a therapy that comprises utilizing an immune response to target and treat a tumor in a subject. As such, as used herein, an I-O therapy is a type of anti-cancer therapy. In some aspects, and I-O therapy comprises administering an antibody to a subject. In some aspects, an I-O therapy comprises administering to a subject an immune cell, e.g., a T cell, e.g., a modified T cell, e.g., a T cell modified to express a chimeric antigen receptor or an particular T cell receptor. In some aspects, the I-O therapy comprises administering a therapeutic vaccine to a subject. In some aspects, the I-O therapy comprises administering a cytokine or a chemokine to a subject. In some aspects, the I-O therapy comprises administering an interleukin to a subject. In some aspects, the I-O therapy comprises administering an interferon to a subject. In some aspects, the I-O therapy comprises administering a colony stimulating factor to a subject.

An “immune response” refers to the action of a cell of the immune system (for example, T lymphocytes, B lymphocytes, natural killer (NK) cells, macrophages, eosinophils, mast cells, dendritic cells, and neutrophils), and soluble macromolecules produced by any of these cells or the liver (including antibodies, cytokines, and complement) that results in selective targeting, binding to, damage to, destruction of, and/or elimination from a vertebrate's body of invading pathogens, cells or tissues infected with pathogens, cancerous or other abnormal cells, or, in cases of autoimmunity or pathological inflammation, normal human cells or tissues.

The term “LAG-3 positive” or “LAG-3 expression positive,” relating to LAG-3 expression, refers to tumor tissue (e.g., a test tissue sample) that is scored as expressing LAG-3 based on the proportion (i.e., percentage) of immune cells (e.g., tumor-infiltrating lymphocytes such as CD8+ T cells) expressing LAG-3 (e.g., greater than or equal to 1% expression).

“LAG-3 negative” or “LAG-3 expression negative,” refers to tumor tissue (e.g., a test tissue sample) that is not scored as expressing LAG-3 (e.g., less than 1% LAG-3 expression).

The term “PD-L1 positive” or “PD-L1 expression positive,” relating to cell surface PD-L1 expression, refers to tumor tissue (e.g., a test tissue sample) that is scored as expressing PD-L1 based on the proportion (i.e., percentage) of tumor cells expressing PD-L1 (e.g., greater than or equal to 1% expression).

The term “PD-L1 negative” or “PD-L1 expression negative” refers to tumor tissue (e.g., a test tissue sample) that is not scored as expressing PD-L1 (e.g., less than 1% expression).

As used herein, “Eastern Cooperative Oncology Group (ECOG) Performance Status (PS)” is a numbering scale used to define the population of patients to be studied in a trial, so that it can be uniformly reproduced among physicians who enroll patients.

Various aspects of the invention are described in further detail in the following subsections.

### II. Methods of the Invention

Provided herein are methods of treating unresectable or metastatic melanoma in a human patient, the methods comprising administering to the patient a LAG-3 antagonist (e.g., an anti-LAG-3 antibody) alone or in combination with a CTLA-4 inhibitor (e.g., an anti-CTLA-4 antibody). In some aspects, the methods comprise further administering one or more additional therapeutic agents (e.g., a PD-1 inhibitor such as an anti-PD-1 antibody) and/or therapies (e.g., a chemotherapy).

In some aspects, the method comprises administering a LAG-3 antagonist and a CTLA-4 inhibitor, wherein the patient has a sensitizing mutation for a targeted inhibitor therapy (e.g., a BRAF mutation).

In some aspects, the method comprises administering a LAG-3 antagonist and a CTLA-4 inhibitor, wherein the patient has received a prior PD-1 pathway inhibitor as a treatment for melanoma.

In some aspects, the method is a first line (1L) therapy.

In some aspects, the method is a second line (2L) therapy.

In some aspects, the method is a third line (3L) therapy.

In some aspects, the patient has progressed on a prior therapy (e.g., a standard of care therapy). Standard of care therapies for different types of cancer are well known by persons of skill in the art. For example, the National Comprehensive Cancer Network (NCCN), an alliance of 21 major cancer centers in the USA, publishes the NCCN Clinical Practice Guidelines in Oncology (NCCN GUIDELINES®) that provide detailed up-to-date information on the standard of care treatments for a wide variety of cancers. See https://www.nccn.org/professionals/physician_gls/default.aspx, last accessed Nov. 4, 2020.

In some aspects, the patient has not received a prior systemic therapy for cancer, the patient has not received a prior systemic therapy for melanoma, or the patient has not received a prior systemic therapy for unresectable or metastatic melanoma.

In some aspects, the patient is naïve to prior immuno-oncology (I-O) therapy. In some aspects, the patient has never received I-O therapy, has received I-O therapy for a cancer other than melanoma, or has received I-O therapy for a previous melanoma but not a current melanoma. In some aspects, the patient is naïve to prior I-O therapy, the patient is naïve to prior I-O therapy for melanoma, or the melanoma is naïve to prior I-O therapy. In some aspects, the prior I-O therapy is an antibody. In some aspects, the antibody binds to a checkpoint inhibitor. In some aspects, the prior I-O therapy is an anti-PD-1 antibody.

In some aspects, a method of the disclosure increases duration of progression-free survival (PFS), objective response rate (ORR), overall survival (OS), or any combination thereof as compared to a standard of care therapy and/or a prior therapy such as disclosed herein.

In certain aspects, a method of the disclosure extends progression-free survival as compared to a standard of care therapy by over: about 12 months, about 13 months, about 14 months, about 15 months, about 16 months, about 17 months, about 18 months, about 2 years, about 3 years, about 4 years, about 5 years, about 6 years, about 7 years, about 8 years, about 9 years, or about 10 years.

In some aspects, a method of the disclosure reduces the size of a tumor, inhibits growth of a tumor, eliminates a tumor from the patient, prevents relapse of melanoma, induces remission of melanoma, provides a complete response or partial response, or any combination thereof.

In some aspects, a method of the disclosure reduces tumor size at least by about 10%, about 20%, about 30%, about 40%, about 50%, about 60%, about 70%, about 80%, about 90%, or about 100% as compared to the tumor size prior to the administration.

In some aspects, the methods of the disclosure comprise administering to the patient a LAG-3 antagonist based on the patient's cancer stage and/or performance status. Cancer stage and/or performance status can be indicated by any one or more systems in the art.

In some aspects, the melanoma is staged based on a tumor/node/metastasis (TNM) staging system such as the American Joint Committee on Cancer (AJCC) classification.

In some aspects, the patient has stage I melanoma, also known as melanoma in situ. In stage I, the cancer is confined to the epidermis. It has not spread to nearby lymph nodes or to distant parts of the body. In some aspects, the patient has histologically confirmed unresectable stage I melanoma.

In some aspects, the patient has stage II melanoma. In stage II, the tumor is more than 1 mm thick and can be thicker than 4 mm. It can be ulcerated or not. The cancer has not spread to nearby lymph nodes or to distant parts of the body. In some aspects, the patient has histologically confirmed unresectable stage II melanoma.

In some aspects, the patient has stage III melanoma. In some aspects, the patient has histologically confirmed unresectable stage III melanoma. Stage III is divided into stages IIIA, IIIB, IIIC, and IIID.

In stage IIIA, the tumor is no more than 2 mm thick and can be ulcerated or not. The cancer has spread to 1 to 3 nearby lymph nodes, but it is so small that it is only seen under the microscope. It has not spread to distant parts of the body. In some aspects, the patient has histologically confirmed unresectable stage IIIA melanoma.

In stage IIIB, (1) there is no sign of the primary tumor and: (a) the cancer has spread to only one nearby lymph node, or (b) the cancer has spread to very small areas of nearby skin (satellite tumors) or to skin lymphatic channels around the tumor (without reaching the nearby lymph nodes), or (2) the tumor is no more than 4 mm thick and can be ulcerated or not and: (a) the cancer has spread to only one nearby lymph node, or (b) the cancer has spread to very small areas of nearby skin (satellite tumors) or to skin lymphatic channels around the tumor (without reaching the nearby lymph nodes), or (c) the cancer has spread to 2 or 3 nearby lymph nodes. In stage IIIB, the cancer has not spread to distant parts of the body. In some aspects, the patient has histologically confirmed unresectable stage IIIB melanoma.

In stage IIIC, (1) there is no sign of the primary tumor, and: (a) the cancer has spread to 2 or more nearby lymph nodes, at least one of which could be seen or felt, or (b) the cancer has spread to very small areas of nearby skin (satellite tumors) or to skin lymphatic channels around the tumor, and it has reached the nearby lymph nodes, or (c) the cancer has spread to nearby lymph nodes that are clumped together, or (2) the tumor is no more than 4 mm thick, and can be ulcerated or not, and: (a) the cancer has spread to very small areas of nearby skin (satellite tumors) or to skin lymphatic channels around the tumor, and it has reached nearby lymph nodes, or (b) the cancer has spread to 4 or more nearby lymph nodes, or it has spread to nearby lymph nodes that are clumped together, or (3) the tumor is more than 2 mm but no more than 4 mm thick and is ulcerated or it is thicker than 4 mm but is not ulcerated, and: (a) the cancer has spread to one or more nearby lymph nodes, and/or (b) the cancer has spread to very small areas of nearby skin (satellite tumors) or to skin lymphatic channels around the tumor, or (4) the tumor is thicker than 4 mm and is ulcerated, and: (a) the cancer has spread to 1 to 3 nearby lymph nodes, which are not clumped together, or (b) the cancer has spread to very small areas of nearby skin (satellite tumors) or to skin lymphatic channels around the tumor, and it may or may not have reached 1 nearby lymph node. In stage IIIC, the cancer has not spread to distant parts of the body. In some aspects, the patient has histologically confirmed unresectable stage IIIC melanoma.

In stage HID, the tumor is thicker than 4 mm and is ulcerated, and: (a) the cancer has spread to 4 or more nearby lymph nodes, or (b) the cancer has spread to nearby lymph nodes that are clumped together, or (c) the cancer has spread to very small areas of nearby skin (satellite tumors) or to skin lymphatic channels around the tumor, and the cancer has spread to at least 2 nearby lymph nodes, or to lymph nodes that are clumped together. In stage HID, the cancer has not spread to distant parts of the body.

In stage IV, the tumor can be any thickness, can be ulcerated or not, and may or may not have spread to nearby lymph nodes. In stage IV, the cancer has spread to distant lymph nodes or to organs such as the lungs, liver, or brain. In some aspects, the patient has histologically confirmed stage IV melanoma.

In some aspects, the patient has histologically confirmed unresectable stage III or stage IV melanoma.

In some aspects, performance status is indicated by Eastern Cooperative Oncology Group performance status (ECOG PS), which utilizes standardized criteria for measuring how disease impacts a patient's daily living abilities. Example definitions for ECOG PS include: “0” for a patient who is fully active and able to carry on all pre-disease performance without restriction; “1” for a patient who is restricted in physically strenuous activity but ambulatory and able to carry out work of a light or sedentary nature; “2” for a patient who is ambulatory and capable of all self-care, up and about more than 50% of waking hours, but unable to carry out any work activities; “3” for a patient who is capable of only limited self-care and is confined to a bed or chair more than 50% of waking hours; and “4” for a patient who is completely disabled, cannot carry on any self-care, and is totally confined to bed or chair.

In some aspects the patient has an ECOG PS of 0, 1, 2, 3, or 4. In some aspects, the patient has an ECOG PS of ≤3. In some aspects, the patient has an ECOG PS of ≤2. In some aspects, the patient has an ECOG PS of ≤1. In some aspects, the patient has an ECOG PS of 0 or 1.

In some aspects, the patient has a B-rapidly accelerated fibrosarcoma proto-oncogene (BRAF, e.g., a BRAF V600 mutation such as BRAF V600E or BRAF V600K), mitogen-activated extracellular signal-regulated kinase kinase (MEK), neuroblastoma RAS viral oncogene homolog (NRAS), and/or proto-oncogene c-KIT (KIT) mutation sensitive to targeted inhibitor therapy.

In some aspects, the patient has a BRAF mutation. In some aspects, the BRAF mutation is a BRAF V600 mutation. In some aspects, the BRAF mutation is a BRAF V600E mutation. In some aspects, the BRAF mutation is a BRAF V600K mutation.

In some aspects, the patient has no BRAF, MEK, NRAS, and/or KIT mutation sensitive to targeted inhibitor therapy.

In some aspects, the targeted inhibitor therapy comprises a tyrosine kinase inhibitor of BRAF and/or MEK. In some aspects, the targeted inhibitor therapy comprises dabrafenib, vemurafenib, encorafenib, trametinib, cobimetinib, and/or binimetinib.

In one aspect, the invention includes a method of selecting an unresectable or metastatic melanoma in a human patient for immunotherapy, comprising determining the level of LAG-3 and/or PD-L1 expression in a tumor sample.

In one aspect, the invention includes a method of treating unresectable or metastatic melanoma in a human patient, comprising: (a) determining the level of LAG-3 expression, the level of PD-L1 expression, and/or the level of LAG-3 and PD-L1 expression in a tumor sample; and (b) administering to the patient a therapeutically effective amount of a LAG-3 antagonist alone or in combination with a therapeutically effective amount of a CTLA-4 inhibitor. In some aspects, the method comprises further administering one or more additional therapeutic agents and/or therapies (e.g., a chemotherapy).

In some aspects, one or more immune cells in tumor tissue from the patient express LAG-3 (i.e., tumor tissue from the patient is LAG-3 positive) and/or one or more tumor cells in tumor tissue from the patient express PD-L1 (i.e., tumor tissue from the patient is PD-L1 positive). In some aspects, one or more immune cells in tumor tissue from the patient express LAG-3. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the immune cells express LAG-3. In some aspects, at least about 1% of the immune cells express LAG-3. In some aspects, greater than about 1% of the immune cells express LAG-3. In some aspects, the immune cells are tumor-infiltrating lymphocytes. In some aspects, the tumor-infiltrating lymphocytes are CD8⁺ cells. In some aspects, one or more tumor cells in tumor tissue from the patient express PD-L1. In some aspects, at least about 1%, at least about 2%, at least about 3%, at least about 4%, at least about 5%, at least about 7%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or about 100% of the tumor cells express PD-L1. In some aspects, at least about 1% of the tumor cells express PD-L1. In some aspects, greater than about 1% of the tumor cells express PD-L1. In some aspects, any of the values of “at least about X %” is “≥X %”).

In some aspects, one or more immune cells in tumor tissue from the patient does not express LAG-3 (i.e., tumor tissue from the patient is LAG-3 negative). In some aspects, the tumor tissue is LAG-3 negative when less than about 1% of the immune cells express LAG-3.

In some aspects, one or more tumor cells in tumor tissue from the patient does not express PD-L1 (i.e., tumor tissue from the patient is PD-L1 negative). In some aspects, the tumor tissue is PD-L1 negative when less than about 1% of the tumor cells express PD-L1.

In some aspects, a method of the disclosure comprises identifying the patient as having immune cells (e.g., tumor infiltrating lymphocytes) and/or tumor cells that express or contain a particular marker. For example, in some aspects, the melanoma is LAG-3 positive. In some aspects, the melanoma is PD-L1 positive. In some aspects, the melanoma is LAG-3 positive and PD-L1 positive. In some aspects, the melanoma contains a BRAF V600 mutation. In some aspects, the melanoma is LAG-3 positive and expresses the BRAF V600 mutation. In some aspects, the melanoma is LAG-3 positive and contains tumor cells that express wild-type BRAF. In some aspects, the melanoma is LAG-3 positive, PD-L1 positive, and contains wild-type BRAF. In some aspects, the melanoma is LAG-3 positive, PD-L1 positive, and contains a BRAF V600 mutation. In some aspects, the melanoma is PD-L1 positive and contains a wild-type BRAF. In some aspects, the melanoma is PD-L1 positive and contains a BRAF V600 mutation.

The invention can also include a method of preventing a relapse and/or inducing a remission in a patient comprising administering to the patient an immunotherapy disclosed herein.

In other aspects, each patient in the methods experiences (i) extended progression-free survival for over 12 months, (ii) tumor size reduction at least about 10%, about 20%, about 30%, about 40%, or about 50% or higher compared to the tumor size prior to the administration, or (iii) both.

The methods of the invention, as a result of the administration of an immunotherapy disclosed herein, can treat unresectable or metastatic melanoma, reduce the tumor size, inhibit growth of the tumor, eliminate the tumor from the patient, prevent a relapse of a tumor, induce a remission in a patient, or any combination thereof. In certain aspects, the administration of an immunotherapy disclosed herein induces a complete response. In other aspects, the administration of the immunotherapy disclosed herein induces a partial response.

In some aspects, LAG-3 and/or PD-L1 expression is determined by receiving the results of an assay capable of determining LAG-3 and/or PD-L1 expression.

Methods for determining PD-L1 expression in a tumor sample, methods for identifying the patient as having a PD-L1 positive malignant tumor, and methods for determining PD-L1 expression in a malignant tumor have been disclosed in PCT/US2016/029878, the teachings of which are hereby incorporated by reference.

In order to assess expression of LAG-3 and/or PD-L1, and/or whether the cancer contains a BRAF V600 mutation, in one aspect, a test tissue sample is obtained from the patient. In some aspects, a test tissue sample includes, but is not limited to, any clinically relevant tissue sample, such as a tumor biopsy, a core biopsy tissue sample, an incisional biopsy, an excisional biopsy, a surgical specimen, a fine needle aspirate, or a sample of bodily fluid, such as blood, plasma, serum, lymph, ascites fluid, cystic fluid, or urine. In some aspects, the test tissue sample is from a primary tumor. In some aspects, the test tissue sample is from a metastasis. In some aspects, test tissue samples are taken from a patient at multiple time points, for example, before treatment, during treatment, and/or after treatment. In some aspects, test tissue samples are taken from different locations in the patient, for example, a sample from a primary tumor and a sample from a metastasis in a distant location.

In some aspects, the test tissue sample is a paraffin-embedded fixed tissue sample. In some aspects, the test tissue sample is a formalin-fixed paraffin embedded (FFPE) tissue sample. In some aspects, the test tissue sample is a fresh tissue (e.g., tumor) sample. In some aspects, the test tissue sample is a frozen tissue sample. In some aspects, the test tissue sample is a fresh frozen (FF) tissue (e.g., tumor) sample. In some aspects, the test tissue sample is a cell isolated from a fluid. In some aspects, the test tissue sample comprises circulating tumor cells (CTCs). In some aspects, the test tissue sample comprises tumor-infiltrating lymphocytes (TILs). In some aspects, the test tissue sample comprises tumor cells and tumor-infiltrating lymphocytes (TILs). In some aspects, the test tissue sample comprises circulating lymphocytes. In some aspects, the test tissue sample is an archival tissue sample. In some aspects, the test tissue sample is an archival tissue sample with known diagnosis, treatment, and/or outcome history. In some aspects, the sample is a block of tissue. In some aspects, the test tissue sample is dispersed cells. In some aspects, the sample size is from about 1 cell to about 1×10⁶ cells or more. In some aspects, the sample size is about 1 cell to about 1×10⁵ cells. In some aspects, the sample size is about 1 cell to about 10,000 cells. In some aspects, the sample size is about 1 cell to about 1,000 cells. In some aspects, the sample size is about 1 cells to about 100 cells. In some aspects, the sample size is about 1 cell to about 10 cells. In some aspects, the sample size is a single cell. In aspects, the assessment of LAG-3, PD-L1, and/or BRAF V600 status is based on circulating tumor DNA.

In another aspect, the assessment of LAG-3, PD-L1, and/or BRAF V600 status can be achieved without obtaining a test tissue sample. In some aspects, selecting a suitable patient includes (i) optionally providing a test tissue sample obtained from a patient with cancer of the tissue, the test tissue sample comprising tumor cells and/or tumor-infiltrating inflammatory cells; and (ii) assessing the proportion of cells in the test tissue sample that express LAG-3, PD-L1, and/or BRAF V600 based on an assessment that the proportion of cells in the test tissue sample is higher than a predetermined threshold level.

In any of the methods comprising the measurement of LAG-3, PD-L1, and/or BRAF V600 status in a test tissue sample, however, it should be understood that the step comprising the provision of a test tissue sample obtained from a patient is an optional step. That is, in certain aspects the method includes this step, and in other aspects, this step is not included in the method. It should also be understood that in certain aspects the “measuring” or “assessing” step to identify, or determine the number or proportion of, cells in the test tissue sample that express LAG-3 and/or PD-L1 is performed by a transformative method of assaying for LAG-3 and/or PD-L1, for example by performing a reverse transcriptase-polymerase chain reaction (RT-PCR) assay or an IHC assay. In certain other aspects, no transformative step is involved and LAG-3 and/or PD-L1 expression is assessed by, for example, reviewing a report of test results from a laboratory. In some aspects, LAG-3 and/or PD-L1 expression is assessed by reviewing the results of an immunohistochemistry assay from a laboratory. In certain aspects, the steps of the methods up to, and including, assessing LAG-3 and/or PD-L1 expression provides an intermediate result that can be provided to a physician or other healthcare provider for use in selecting a suitable candidate for a method of the disclosure. In certain aspects, the steps that provide the intermediate result is performed by a medical practitioner or someone acting under the direction of a medical practitioner. In other aspects, these steps are performed by an independent laboratory or by an independent person such as a laboratory technician. In some aspects, the presence of a BRAF V600 mutation is performed using parallel approaches for LAG-3 and/or PD-L1.

In certain aspects of any of the present methods, the proportion of cells that express LAG-3 and/or PD-L1, and/or contain a BRAF V600 mutation, is assessed by performing an assay to detect the presence of LAG-3, PD-L1, and/or BRAF RNA. In further aspects, the presence of LAG-3, PD-L1, and/or BRAF RNA is detected by RT-PCR, in situ hybridization, or RNase protection. In some aspects, the presence of LAG-3, PD-L1, and/or BRAF RNA is detected by an RT-PCR based assay. In some aspects, scoring the RT-PCR based assay comprises assessing the level of LAG-3, PD-L1, and/or BRAF RNA expression in the test tissue sample relative to a predetermined level. In some aspects, expression of one or more of LAG-3, PD-L1, and BRAF V600 is assessed using gene expression profiling.

In other aspects, the proportion of cells that express LAG-3 and/or PD-L1, and/or contain a BRAF V600 mutation, is assessed by performing an assay to detect the presence of LAG-3 and/or PD-L1, and/or contain a BRAF V600 mutation polypeptide. In further aspects, the presence of LAG-3, PD-L1, and/or BRAF V600 polypeptide is detected by IHC, enzyme-linked immunosorbent assay (ELISA), in vivo imaging, or flow cytometry. In some aspects, LAG-3 and/or PD-L1 expression and/or BRAF V600 status is assayed by IHC. In other aspects of all of these methods, cell surface expression of LAG-3 and/or PD-L1 and/or the presence of a BRAF V600 mutation is assayed using, e.g., IHC or in vivo imaging.

In other aspects, the proportion of cells that express LAG-3 and/or PD-L1 and/or contain a BRAF V600 mutation in the test tissue sample is assessed by flow cytometry. In some aspects, the test tissue sample assayed by flow cytometry comprises tumor infiltrating immune cells. In some aspects, the flow cytometry is a multiplex assay. In some aspects, scoring the flow cytometry comprises detecting the expression of markers comprising LAG-3, CD4, CD8, FOXP3, and any combination thereof. In some aspects, scoring the flow cytometry comprises assessing the proportion of T cells in the test tissue sample that express LAG-3 and/or PD-L1, and/or contain a BRAF V600 mutation. In some aspects, scoring the flow cytometry comprises assessing the proportion of CD8+ T cells in the test tissue sample that express LAG-3 and/or PD-L1, and/or contain a BRAF V600 mutation. In some aspects, scoring the flow cytometry comprises assessing the proportion of CD4+ T cells in the test tissue sample that express LAG-3 and/or PD-L1, and/or contain a BRAF V600 mutation. In some aspects, scoring the flow cytometry comprises assessing the proportion of FOXP3+ T cells in the test tissue sample that express LAG-3 and/or PD-L1, and/or contain a BRAF V600 mutation.

In certain aspects of any of the present methods, the proportion of cells that express LAG-3, PD-L1, and/or contain a BRAF V600 in the test tissue sample comprises performing an assay to detect the presence of LAG-3, PD-L1, and/or BRAF V600 polypeptide. In some aspects, the presence of LAG-3, PD-L1, and/or BRAF V600 polypeptide is detected by an immunohistochemistry assay. In some aspects, the test tissue sample is a tumor biopsy. In some aspects, the test tissue sample is a formalin-fixed paraffin embedded (FFPE) sample.

In some aspects, the immunohistochemistry assay is a monoplex assay. In some aspects, the immunohistochemistry assay is a multiplex assay. In some aspects, the multiplex immunohistochemistry assay is capable of detecting the presence of CD4, CD8, FOXP3, or any combination thereof.

In some aspects, the immunohistochemistry assay comprises contacting the tumor sample with the 17B4 mouse anti-human LAG-3 IgG1 monoclonal antibody. In some aspects, the immunohistochemistry assay comprises contacting the tumor sample with an anti-LAG-3 antibody comprising heavy and light chain variable regions comprising the sequences set forth in SEQ ID NOs:3 and 5, respectively. In some aspects, the immunohistochemistry assay comprises contacting the tumor sample with the SP346 rabbit anti-human LAG-3 IgG monoclonal antibody. In some aspects, the immunohistochemistry assay comprises contacting the tumor sample with the 11E3 (Novusbio), 874501 (Novusbio), or EPR4392(2) (Abcam) anti-human LAG-3 monoclonal antibody. In some aspects, the immunohistochemistry assay comprises contacting the tumor sample with reagents in the Dako PD-L1 IHC 28-8 kit to assay for PD-L1 expression.

In some aspects, the immunohistochemistry assay is scored at a low magnification. In some aspects, low magnification is about 20×. In some aspects, the immunohistochemistry assay is scored at high magnification. In some aspects, high magnification is about 40×.

In some aspects, the immunohistochemistry assay is scored by an image analysis software. In some aspects, the immunohistochemistry assay is scored by pathologist visual immune score. In some aspects, the immunohistochemistry assay is scored manually.

In some aspects, scoring the immunohistochemistry assay comprises assessing the proportion of cells in the test tissue sample that express LAG-3 and/or PD-L1, and/or contain a BRAF V600 mutation. In some aspects, scoring the immunohistochemistry assay comprises assessing the proportion of immune cells in the test tissue sample that express LAG-3 and/or PD-L1, and/or contain a BRAF V600 mutation. In some aspects, scoring the immunohistochemistry assay comprises assessing the proportion of T cells in the test tissue sample that express LAG-3 and/or PD-L1, and/or contain a BRAF V600 mutation. In some aspects, scoring the immunohistochemistry assay comprises assessing the proportion of CD8+ T cells in the test tissue sample that express LAG-3. In some aspects, scoring the immunohistochemistry assay comprises assessing the proportion of CD4+ T cells in the test tissue sample that express LAG-3. In some aspects, scoring the immunohistochemistry assay comprises assessing the proportion of FOXP3+ T cells in the test tissue sample that express LAG-3.

In some aspects, the immunohistochemistry assay is a multiplex assay that further comprises detecting the expression of MHC Class II by the tumor cells. In some aspects, scoring the immunohistochemistry assay comprises assessing the proportion of cells in the test tissue sample that expresses MHC Class II. In some aspects, scoring the immunohistochemistry assay comprises assessing the proportion of non-immune cells in the test tissue sample that expresses MHC Class II.

In a particular aspects, the expression of fibrinogen-like protein 1 (FGL1) by the tumor cells is measured.

Imaging techniques have provided important tools in cancer research and treatment. Recent developments in molecular imaging systems, including positron emission tomography (PET), single-photon emission computed tomography (SPECT), fluorescence reflectance imaging (FRI), fluorescence-mediated tomography (FMT), bioluminescence imaging (BLI), laser-scanning confocal microscopy (LSCM) and multiphoton microscopy (MPM), will likely herald even greater use of these techniques in cancer research. Some of these molecular imaging systems allow clinicians to not only see where a tumor is located in the body, but also to visualize the expression and activity of specific molecules, cells, and biological processes that influence tumor behavior and/or responsiveness to therapeutic drugs (Condeelis and Weissleder, Cold Spring Harb. Perspect. Biol. 2(12):a003848 (2010)). Antibody specificity, coupled with the sensitivity and resolution of PET, makes immunoPET imaging particularly attractive for monitoring and assaying expression of antigens in tissue samples (McCabe and Wu, Cancer Biother. Radiopharm. 25(3):253-61 (2010); Olafsen et al., Protein Eng. Des. Sel. 23(4):243-9 (2010)). In certain aspects of any of the present methods, LAG-3, PD-L1, and/or BRAF V600 expression is assayed by immunoPET imaging. In certain aspects of any of the present methods, the proportion of cells in a test tissue sample that express LAG-3, PD-L1, and/or BRAF V600 is assessed by performing an assay to determine the presence of LAG-3, PD-L1, and/or BRAF V600 polypeptide on the surface of cells in the test tissue sample. In certain aspects, the test tissue sample is a FFPE tissue sample. In other aspects, the presence of LAG-3, PD-L1 and/or BRAF V600 polypeptide is determined by IHC assay. In further aspects, the IHC assay is performed using an automated process.

In some aspects, the presence of BRAF V600E mutation in a tumor specimen is confirmed prior to initiation of treatment. In certain aspects, the BRAF V600E mutation is confirmed by an FDA-approved test. In a particular aspect, the test to confirm the presence of the BRAF V600E mutation is the Cobas® 4800 BRAF V600 Mutation Test.

II.A. Assaying LAG-3 and/or PD-L1 Expression and/or the Presence of a BRAF V600 Mutation by Automated IHC

In one aspect of the present methods, an automated IHC method is used to assay the expression of LAG-3 and/or PD-L1, and/or the presence a BRAF V600 mutation in FFPE tissue specimens. This disclosure provides methods for detecting the presence of human LAG-3, PD-L1, and/or BRAF V600 in a test tissue sample, or quantifying the level of human LAG-3, PD-L1, and/or BRAF V600 antigen or the proportion of cells in the sample that express the antigen, which methods comprise contacting the test sample, and a negative control sample, with a mAb that specifically binds to human LAG-3, PD-L1, and/or BRAF V600, under conditions that allow for formation of a complex between the antibody or portion thereof and human LAG-3, PD-L1, and/or BRAF V600. In certain aspects, the test and control tissue samples are FFPE samples. The formation of a complex is then detected, wherein a difference in complex formation between the test sample and the negative control sample is indicative of the presence of human LAG-3, PD-L1, and/or BRAF V600 antigen in the sample. Various methods are used to quantify LAG-3, PD-L1, and/or BRAF V600.

In a particular aspect, the automated IHC method comprises: (a) deparaffinizing and rehydrating mounted tissue sections in an autostainer; (b) retrieving antigen in an autostainer; (c) setting up reagents on an autostainer; and (d) running the autostainer to include steps of neutralizing endogenous peroxidase in the tissue specimen; blocking non-specific protein-binding sites on the slides; incubating the slides with primary Ab; incubating with a postprimary blocking agent; incubating with a postprimary antibody detection agent, such as another antibody that may or may not be conjugated to a detection enzyme; incubating with a polymeric-enzyme detection reagent; adding a chromogen substrate and developing; and counterstaining with hematoxylin. In some aspects, the retrieving antigen comprises using any heat based antigen retrieval device.

In some aspects, for assessing the presence of LAG-3, PD-L1, and/or BRAF V600 in tumor tissue samples, a pathologist examines the number of LAG-3+ tumor infiltrating lymphocytes, PD-L1+ tumor cells, and/or BRAF V600+ tumor cells in each field under a microscope and mentally estimates the percentage of cells that are positive, then averages them to come to the final percentage. The different staining intensities are defined as 0/negative, 1+/weak, 2+/moderate, and 3+/strong. Typically, percentage values are first assigned to the 0 and 3+ buckets, and then the intermediate 1+ and 2+ intensities are considered. For highly heterogeneous tissues, the specimen is divided into zones, and each zone is scored separately and then combined into a single set of percentage values. The percentages of negative and positive cells for the different staining intensities are determined from each area and a median value is given to each zone. A final percentage value is given to the tissue for each staining intensity category: negative, 1+, 2+, and 3+. The sum of all staining intensities needs to be 100%.

In some aspects, staining is also assessed in tumor-infiltrating inflammatory cells such as macrophages and lymphocytes. Macrophages and lymphocytes are assessed for LAG-3, PD-L1, and/or BRAF V600 staining and only recorded for all samples as being positive or negative for each cell category. Staining is also characterized according to an outside/inside tumor immune cell designation. “Inside” means the immune cell is within the tumor tissue and/or on the boundaries of the tumor region without being physically intercalated among the tumor cells. “Outside” means that there is no physical association with the tumor, the immune cells being found in the periphery associated with connective or any associated adjacent tissue.

In certain aspects of these scoring methods, the samples are scored by two or more pathologists operating independently, and the scores are subsequently consolidated. In certain other aspects, the identification of positive and negative cells is scored using appropriate software.

A histoscore (H-score) is used as a more quantitative measure of the IHC data. The histoscore is calculated as follows:

$$Histoscore = [ ( \% tumor \times 1 ( low intensity ) ) + ( \% tumor \times 2 ( medium intensity ) ) + ( \% tumor \times 3 ( high intensity ) ]$$

To determine the histoscore, the pathologist estimates the percentage of stained cells in each intensity category within a specimen. Because expression of most biomarkers is heterogeneous the histoscore is a truer representation of the overall expression. The final histoscore range is 0 (minimum score, no expression) to 300 (maximum score, strong and inclusive expression).

### II.B. LAG-3 Antagonists

A LAG-3 antagonist for use in the methods of the disclosure includes, but is not limited to, LAG-3 binding agents and soluble LAG-3 polypeptides. LAG-3 binding agents include antibodies that specifically bind to LAG-3 (i.e., an “anti-LAG-3 antibody”). The term “LAG-3 antagonist” as used herein is interchangeable with the term “LAG-3 inhibitor.”

In some aspects, the LAG-3 antagonist is an anti-LAG-3 antibody.

Antibodies that bind to LAG-3 have been disclosed, for example, in Int'l Publ. No. WO/2015/042246 and U.S. Publ. Nos. 2014/0093511 and 2011/0150892, each of which is incorporated by reference herein in its entirety.

An exemplary LAG-3 antibody useful in the present disclosure is 25F7 (described in U.S. Publ. No. 2011/0150892). An additional exemplary LAG-3 antibody useful in the present disclosure is BMS-986016 (relatlimab). In some aspects, an anti-LAG-3 antibody useful in the present disclosure cross-competes with 25F7 or BMS-986016. In some aspects, an anti-LAG-3 antibody useful in the present disclosure binds to the same epitope as 25F7 or BMS-986016. In some aspects, an anti-LAG-3 antibody comprises six CDRs of 25F7 or BMS-986016.

Other art-recognized anti-LAG-3 antibodies that can be used in the methods of the disclosure include IMP731 (H5L7BW) described in US 2011/007023, MK-280 (28G-10) described in WO2016028672 and U.S. Publication No. 2020/0055938, REGN3767 (fianlimab) described in Burova E, et al., J. Immunother. Cancer (2016); 4(Supp. 1):P195 and U.S. Pat. No. 10,358,495, humanized BAP050 described in WO2017/019894, GSK2831781, IMP-701 (LAG525; ieramilimab) described in U.S. Pat. No. 10,711,060 and U.S. Publ. No. 2020/0172617, aLAG3(0414), aLAG3(0416), Sym022, TSR-033, TSR-075, XmAb22841, MGD013, B1754111, FS118, P 13B02-30, AVA-017 and AGEN1746. These and other anti-LAG-3 antibodies useful in the claimed invention can be found in, for example: U.S. Pat. No. 10,188,730, WO 2016/028672, WO 2017/106129, WO2017/062888, WO2009/044273, WO2018/069500, WO2016/126858, WO2014/179664, WO2016/200782, WO2015/200119, WO2017/019846, WO2017/198741, WO2017/220555, WO2017/220569, WO2018/071500, WO2017/015560, WO2017/025498, WO2017/087589, WO2017/087901, WO2018/083087, WO2017/149143, WO2017/219995, US2017/0260271, WO2017/086367, WO2017/086419, WO2018/034227, WO2018/185046, WO2018/185043, WO2018/217940, WO19/011306, WO2018/208868, WO2014/140180, WO2018/201096, WO2018/204374, and WO2019/018730. The contents of each of these references are incorporated by reference in their entirety.

Anti-LAG-3 antibodies that can be used in the methods of the disclosure also include isolated antibodies that bind specifically to human LAG-3 and cross-compete for binding to human LAG-3 with any anti-LAG-3 antibody disclosed herein, e.g., relatlimab. In some aspects, the anti-LAG-3 antibody binds the same epitope as any of the anti-LAG-3 antibodies described herein, e.g., relatlimab.

In some aspects, the antibodies that cross-compete for binding to human LAG-3 with, or bind to the same epitope region as, any anti-LAG-3 antibody disclosed herein, e.g., relatlimab, are monoclonal antibodies. For administration to human subjects, these cross-competing antibodies are chimeric antibodies, engineered antibodies, or humanized or human antibodies. Such chimeric, engineered, humanized, or human monoclonal antibodies can be prepared and isolated by methods well known in the art.

The ability of antibodies to cross-compete for binding to an antigen indicates that the antibodies bind to the same epitope region of the antigen and sterically hinder the binding of other cross-competing antibodies to that particular epitope region. These cross-competing antibodies are expected to have functional properties very similar those of the reference antibody, e.g., relatlimab, by virtue of their binding to the same epitope region. Cross-competing antibodies can be readily identified based on their ability to cross-compete in standard binding assays such as Biacore analysis, ELISA assays or flow cytometry (see, e.g., WO 2013/173223).

Anti-LAG-3 antibodies that can be used in the methods of the disclosure also include antigen-binding portions of any of the above full-length antibodies. It has been amply demonstrated that the antigen-binding function of an antibody can be performed by fragments of a full-length antibody.

In some aspects, the anti-LAG-3 antibody is a full-length antibody.

In some aspects, the anti-LAG-3 antibody is a monoclonal, human, humanized, chimeric, or multispecific antibody. In some aspects, the multispecific antibody is a dual-affinity re-targeting antibody (DART), a DVD-Ig, or bispecific antibody.

In some aspects, the anti-LAG-3 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-LAG-3 antibody is BMS-986016 (relatlimab), IMP731 (H5L7BW), MK4280 (28G-10), REGN3767 (fianlimab), GSK2831781, humanized BAP050, IMP-701 (LAG525, ieramilimab), aLAG3(0414), aLAG3(0416), Sym022, TSR-033, TSR-075, XmAb22841, MGD013, BI754111, FS118, P 13B02-30, AVA-017, 25F7, AGEN1746, or comprises an antigen binding portion thereof.

In some aspects, the anti-LAG-3 antibody is relatlimab.

In some aspects, the anti-LAG-3 antibody comprises sequences of the heavy and light chain CDRs, heavy and light chain variable regions, or heavy and light chains of an anti-LAG-3 antibody disclosed herein or as known in the art, such as sequences provided in the publications disclosed herein.

In some aspects, a method as disclosed herein comprises an anti-LAG-3 antibody having at least about 90% sequence identity with an anti-LAG-3 antibody as disclosed herein or as known in the art (e.g., at least about 90%, about 91%, about 92%, about 93%, about 94%, about 95%, about 96%, about 97%, about 98%, or about 99% identity to a sequence of an anti-LAG-3 antibody, such as to the heavy chain variable region and/or light chain variable region or to the heavy chain and/or light chain of an anti-LAG-3 antibody).

In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5.

In some aspects, the anti-LAG-3 antibody comprises: (a) a heavy chain variable region CDR1 comprising the sequence set forth in SEQ ID NO:7; (b) a heavy chain variable region CDR2 comprising the sequence set forth in SEQ ID NO: 8; (c) a heavy chain variable region CDR3 comprising the sequence set forth in SEQ ID NO:9; (d) a light chain variable region CDR1 comprising the sequence set forth in SEQ ID NO:10; (e) a light chain variable region CDR2 comprising the sequence set forth in SEQ ID NO:11; and (f) a light chain variable region CDR3 comprising the sequence set forth in SEQ ID NO:12.

In some aspects, the anti-LAG-3 antibody comprises heavy and light chain variable regions comprising the sequences set forth in SEQ ID NOs:3 and 5, respectively.

In some aspects, the anti-LAG-3 antibody comprises heavy and light chains comprising the sequences set forth in SEQ ID NOs:1 and 2, respectively.

In some aspects, the anti-LAG-3 antibody comprises heavy and light chains comprising the sequences set forth in SEQ ID NOs:30 and 2, respectively.

In some aspects, the anti-LAG-3 antibody is REGN3767 (fianlimab). In some aspects, fianlimab is administered intravenously at a dose of about 1 mg/kg, about 3 mg/kg, about 10 mg/kg, or about 20 mg/kg once about every 3 weeks.

In some aspects, the anti-LAG-3 antibody is LAG525 (ieramilimab). In some aspects, ieramilimab is administered intravenously at a dose of about 300 mg, about 400 mg, about 500 mg, about 600 mg, about 700 mg, about 800 mg, about 900 mg, about 1000 mg, about 1100 mg, about 1200 mg, or about 1300 mg once about every 2, 3, or 4 weeks.

In some aspects, the anti-LAG-3 antibody is MK4280. In some aspects, MK4280 is administered intravenously at a dose of about 7 mg, 21 mg, 70 mg, 210 mg, or 700 mg once about every 3 weeks.

In some aspects, the LAG-3 antagonist is a soluble LAG-3 polypeptide. In some aspects, the soluble LAG-3 polypeptide is a fusion polypeptide, e.g., a fusion protein comprising the extracellular portion of LAG-3. In some aspects, the soluble LAG-3 polypeptide is a LAG-3-Fc fusion polypeptide capable of binding to MHC Class II. In some aspects, the soluble LAG-3 polypeptide comprises a ligand binding fragment of the LAG-3 extracellular domain. In some aspects, the ligand binding fragment of the LAG-3 extracellular domain comprises an amino acid sequence with at least about 90%, at least about 95%, at least about 98%, at least about 99%, or about 100% sequence identity to SEQ ID NO:41. In some aspects, the soluble LAG-3 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble LAG-3 polypeptide is IMP321 (eftilagimod alpha). See, e.g., Brignone C, et al., J. Immunol. (2007); 179:4202-4211 and WO2009/044273.

In some aspects, an anti-LAG-3 antibody is used to determine LAG-3 expression. In some aspects, an anti-LAG-3 antibody is selected for its ability to bind to LAG-3 in formalin-fixed, paraffin-embedded (FFPE) tissue specimens. In some aspects, an anti-LAG-3 antibody is capable of binding to LAG-3 in frozen tissues. In some aspects, an anti-LAG-3 antibody is capable of distinguishing membrane bound, cytoplasmic, and/or soluble forms of LAG-3.

In some aspects, an anti-LAG-3 antibody useful for assaying, detecting, and/or quantifying LAG-3 expression in accordance with the methods disclosed herein is the 17B4 mouse IgG1 anti-human LAG-3 monoclonal antibody. See, e.g., Matsuzaki, J et al., PNAS (2010); 107:7875.

In some aspects, the LAG-3 antagonist is formulated for intravenous administration.

In some aspects, the LAG-3 antagonist is administered at a flat dose.

In some aspects, the LAG-3 antagonist is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the LAG-3 antagonist is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the LAG-3 antagonist is administered at a dose of about 360 mg, about 720 mg, about 1080 mg, or about 1200 mg.

In some aspects, the LAG-3 antagonist is administered at a weight-based dose.

In some aspects, the LAG-3 antagonist is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the LAG-3 antagonist is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, a LAG-3 antagonist as described herein is administered as a monotherapy, i.e., the LAG-3 antagonist is not administered in combination with another therapeutic agent.

In some aspects, a LAG-3 antagonist or a combination of a LAG-3 antagonist and a CTLA-4 inhibitor as described herein is administered with one or more additional therapeutic agents and/or anti-cancer therapies.

### II.C. CTLA-4 Inhibitors

CTLA-4 inhibitors that are known in the art can be used in the methods of the disclosure. In some aspects, a CTLA-4 inhibitor for use in the methods of the disclosure includes, but is not limited to, a CTLA-4 binding agent. In some aspects, a CTLA-4 binding agent binds to human CTLA-4 and disrupts the interaction of CTLA-4 with a human B7 receptor. Because the interaction of CTLA-4 with B7 transduces a signal leading to inactivation of T-cells bearing the CTLA-4 receptor, disruption of the interaction effectively induces, enhances, or prolongs the activation of such T cells, thereby inducing, enhancing or prolonging an immune response.

In some aspects, the CTLA-4 inhibitor is an anti-CTLA-4 antibody.

Human monoclonal antibodies that bind specifically to CTLA-4 with high affinity have been disclosed in U.S. Pat. No. 6,984,720. Other anti-CTLA-4 monoclonal antibodies have been described in, for example, U.S. Pat. Nos. 5,977,318, 6,051,227, 6,682,736, and 7,034,121 and International Publication Nos. WO 2012/122444, WO 2007/113648, WO 2016/196237, and WO 2000/037504, each of which is incorporated by reference herein in its entirety. The anti-CTLA-4 human monoclonal antibodies disclosed in U.S. Pat. No. 6,984,720 have been demonstrated to exhibit one or more of the following characteristics: (a) binds specifically to human CTLA-4 with a binding affinity reflected by an equilibrium association constant (Kₐ) of at least about 10⁷ M⁻¹, or about 10⁹ M⁻¹, or about 10¹⁰ M⁻¹ to 10¹¹ M⁻¹ or higher, as determined by Biacore analysis; (b) a kinetic association constant (kₐ) of at least about 10³, about 10⁴, or about 10⁵ m⁻¹ s⁻¹; (c) a kinetic disassociation constant (k_{d}) of at least about 10³, about 10⁴, or about 10⁵ m⁻¹ s⁻¹; and (d) inhibits the binding of CTLA-4 to B7-1 (CD80) and B7-2 (CD86). Anti-CTLA-4 antibodies useful for the present disclosure include monoclonal antibodies that bind specifically to human CTLA-4 and exhibit at least one, at least two, or at least three of the preceding characteristics.

Anti-CTLA-4 antibodies that can be used in the methods of the disclosure include ipilimumab (also known as YERVOY®, MDX-010, 10D1; see U.S. Pat. No. 6,984,720), MK1308 (Merck, also known as quavonlimab), AGEN1884 (Agenus Inc., also known as zalifrelimab; see WO 2016/196237), tremelimumab (AstraZeneca; also known as ticilimumab, CP-675,206; see WO 2000/037504 and Ribas, Update Cancer Ther. 2(3): 133-39 (2007)), REGN4659 (Regeneron Pharmaceuticals, Inc.; see U.S. Pub. No. 2019/0048096), and CTLA-4 probody BMS-986249 (BMS, an anti-CTLA-4 antibody with a peptide mask that is cleaved off, see WO18/085555).

In some aspects, the anti-CTLA-4 antibody binds specifically to human CTLA-4 and cross-competes for binding to human CTLA-4 with any anti-CTLA-4 antibody disclosed herein, e.g., ipilimumab and/or tremelimumab. In some aspects, the anti-CTLA-4 antibody binds the same epitope as any of the anti-CTLA-4 antibodies described herein, e.g., ipilimumab and/or tremelimumab.

In some aspects, the antibodies that cross-compete for binding to human CTLA-4 with, or bind to the same epitope region as, any anti-CTLA-4 antibody disclosed herein, e.g., ipilimumab and/or tremelimumab, are monoclonal antibodies. For administration to human subjects, these cross-competing antibodies are chimeric antibodies, engineered antibodies, or humanized or human antibodies.

Anti-CTLA-4 antibodies that can be used in the methods of the disclosure also include antigen-binding portions of any of the above full-length antibodies.

In some aspects, the anti-CTLA-4 antibody is a full-length antibody. In some aspects, the anti-CTLA-4 antibody is a monoclonal, human, humanized, chimeric, or multispecific antibody. In some aspects, the multispecific antibody is a DART, a DVD-Ig, or bispecific antibody.

In some aspects, the anti-CTLA-4 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-CTLA-4 antibody is ipilimumab, tremelimumab, MK1308, AGEN1884, REGN4659, or comprises an antigen binding portion thereof.

In some aspects, the CTLA-4 antibody is tremelimumab (also known as CP-675,206). Tremelimumab is human IgG2 monoclonal anti-CTLA-4 antibody. Tremelimumab is described in WO/2012/122444, U.S. Publ. No. 2012/263677, or WO Publ. No. 2007/113648 A2.

In some aspects, the anti-CTLA-4 antibody is ipilimumab. Ipilimumab is a fully human, IgG1 monoclonal antibody that blocks the binding of CTLA-4 to its B7 ligands, thereby stimulating T cell activation.

In some aspects, the anti-CTLA-4 antibody comprises sequences of the heavy and light chain CDRs, heavy and light chain variable regions, or heavy and light chains of an anti-CTLA-4 antibody disclosed herein or as known in the art, such as sequences provided in the publications disclosed herein.

In some aspects, a method as disclosed herein comprises an anti-CTLA-4 antibody having at least about 90% sequence identity with an anti-CTLA-4 antibody as disclosed herein or as known in the art (e.g., at least about 90%, about 91%, about 92%, about 93%, about 94%, about 95%, about 96%, about 97%, about 98%, or about 99% identity to a sequence of an anti-CTLA-4 antibody, such as to the heavy chain variable region and/or light chain variable region or to the heavy chain and/or light chain of an anti-CTLA-4 antibody).

In some aspects, the anti-CTLA-4 antibody comprises the complementarity determining regions (CDRs) of ipilimumab, identified as 10D1 in U.S. Pat. Nos. 6,984,720 and 7,605,238, which are hereby incorporated by reference in their entireties. Ipilimumab (also formerly known as IvtDX-010 and BMS-734016) is marketed as YERVOY® and has been approved for the treatment of metastatic melanoma and is in clinical testing in other cancers. See Hoos et al. (2010) Semin. Oncol. 37:533; Hodi et al. (2010) New Engl J. Med. 363:711; Pardoll (2012) Nat. Immunol. 13(12):1129.

In some aspects, the anti-CTLA-4 antibody comprises heavy and light chain CDRs of ipilimumab, heavy and light chain variable regions of ipilimumab, or heavy and light chains of ipilimumab.

In some aspects, the anti-CTLA-4 antibody comprises CDR1, CDR2, and CDR3 domains of the heavy chain variable region having the sequence set forth in SEQ ID NO:34, and CDR1, CDR2 and CDR3 domains of the light chain variable region having the sequence set forth in SEQ ID NO:32.

In some aspects, the anti-CTLA-4 antibody comprises: (a) a heavy chain variable region CDR1 comprising the sequence set forth in SEQ ID NO:35; (b) a heavy chain variable region CDR2 comprising the sequence set forth in SEQ ID NO:36; (c) a heavy chain variable region CDR3 comprising the sequence set forth in SEQ ID NO:37; (d) a light chain variable region CDR1 comprising the sequence set forth in SEQ ID NO:38; (e) a light chain variable region CDR2 comprising the sequence set forth in SEQ ID NO:39; and (f) a light chain variable region CDR3 comprising the sequence set forth in SEQ ID NO:40.

In some aspects, the anti-CTLA-4 antibody comprises heavy and/or light chain variable regions comprising the sequences set forth in SEQ ID NO:34 and/or SEQ ID NO:32, respectively.

In some aspects, the anti-CTLA-4 antibody comprises heavy and light chain variable regions comprising the sequences set forth in SEQ ID NO:34 and SEQ ID NO:32, respectively.

In some aspects, the anti-CTLA-4 antibody comprises the heavy and light chains of ipilimumab.

In some aspects, the CTLA-4 inhibitor (e.g., an anti-CTLA-4 antibody) is formulated for intravenous administration.

In some aspects, the CTLA-4 inhibitor is administered at a flat dose.

In some aspects, the CTLA-4 inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the CTLA-4 inhibitor is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the CTLA-4 inhibitor is administered at a weight-based dose.

In some aspects, the CTLA-4 inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the CTLA-4 inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the CTLA-4 inhibitor is ipilimumab and is administered at a dose of about 3 mg/kg once about every 3 weeks, about 10 mg/kg once about every 3 weeks, or about 10 mg/kg once about every 12 weeks. In some aspects, ipilimumab is administered for four doses.

In some aspects, the LAG-3 antagonist and the CTLA-4 inhibitor are formulated separately.

In some aspects, the LAG-3 antagonist and the CTLA-4 inhibitor are formulated together.

In some aspects, the LAG-3 antagonist is administered before the CTLA-4 inhibitor.

In some aspects, the CTLA-4 inhibitor is administered before the LAG-3 antagonist.

In some aspects, the LAG-3 antagonist and the CTLA-4 inhibitor are administered concurrently.

### II.D. Anti-Cancer Therapies and Therapeutic Agents

In some aspects, a LAG-3 antagonist as described herein or the combination of a LAG-3 antagonist and a CTLA-4 inhibitor as described herein is administered with one or more additional anti-cancer therapies and/or therapeutic agents.

The additional therapeutic agent and/or anti-cancer therapy can comprise any known therapeutic agent or anti-cancer therapy, including a standard of care in the art for the treatment of a patient afflicted with melanoma.

In some aspects, the additional anti-cancer therapy comprises a surgery, a radiation therapy, a chemotherapy, an immunotherapy, or any combination thereof. In some aspects, the additional anti-cancer therapy comprises a chemotherapy, including any chemotherapeutic agent disclosed herein. In some aspects, the chemotherapy comprises platinum doublet chemotherapy.

In some aspects, the additional therapeutic agent comprises an anti-cancer agent. In some aspects, the anti-cancer agent comprises a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

In some aspects, the tyrosine kinase inhibitor (TKI) comprises sorafenib (e.g., sorafenib tosylate, also known as NEXAVAR®), lenvatinib (e.g., lenvatinib mesylate, also known as LENVIMA®), regorafenib (e.g., STIVARGA®), cabozantinib (e.g., cabozantinib S-malate, also known as CABOMETYX®), sunitinib (e.g., sunitinib malate, also known as SUTENT®), brivanib, linifanib, pemigatinib (also known as PEMAZYRE™), everolimus (also known as AFINITOR® or ZORTRESS®), gefitinib (IRESSA®, a small-molecule TKI of EGFR), imatinib (e.g., imatinib mesylate), lapatinib (e.g., lapatinib ditosylate, also known as TYKERB®), nilotinib (e.g., nilotinib hydrochloride, also known as TASIGNA®), pazopanib (e.g., pazopanib hydrochloride, also known as VOTRIENT®), temsirolimus (also known as TORISEL®), erlotinib (e.g., erlotinib hydrochloride, also known as TARCEVA®, a small-molecule TKI of EGFR), afatinib (GILOTRIF®, a small-molecule TKI of EGFR), dacomitinib (VIZIMPRO®, a small-molecule TKI of EGFR), osimeritinb (TAGRISSO®, a small-molecule TKI of EGFR), alectinib (ALECENSA®, a small-molecule TKI of ALK), ceritinib (ZYKADIA®, a small-molecule TKI of ALK and ROS-1), brigatinib (ALUNBRIG®, a small-molecule TKI of ALK), crizotinib (XALKORI®, a small-molecule TKI of ALK and ROS-1), lorlatinib (LORBRENA®, a small-molecule TKI of ALK and ROS-1), entrectinib (ROZLYTREK®, a small-molecule TKI of ROS-1 and NTRK), larotrectinib (ROZLYTREK®, a small-molecule TKI of NTRK), dabrafenib (TAFINLAR®, a small-molecule TKI of BRAF), vemurafenib (ZELBORAF®, a small-molecule TKI of BRAF), encorafenib (BRAFTOVI®, a small-molecule TKI of BRAF), trametinib (MEKINIST®, a small-molecule TKI of MEK), cobimetinib (COTELLIC®, a small-molecule TKI of MEK), binimetinib (MEKTOVI®, a small-molecule TKI of MEK), or any combination thereof.

In some aspects, the TKI is an inhibitor of BRAF. In some aspects, the TKI is dabrafenib, vemurafenib and/or encorafenib.

In some aspects, the TKI is an inhibitor of MEK. In some aspects, the TKI is trametinib, cobimetinib, and/or binimetinib.

In some aspects, the patient has a BRAF and/or a MEK mutation and the method as disclosed herein further comprises administering a TKI that is an inhibitor of BRAF and/or a TKI that is an inhibitor of MEK as a targeted inhibitor therapy.

In some aspects, the anti-angiogenesis agent comprises an inhibitor of a vascular endothelial growth factor (VEGF), VEGF receptor (VEGFR), platelet-derived growth factor (PDGF), PDGF receptor (PDGFR), angiopoietin (Ang), tyrosine kinase with Ig-like and EGF-like domains (Tie) receptor, hepatocyte growth factor (HGF), tyrosine-protein kinase Met (c-MET), C-type lectin family 14 member A (CLEC14A), multimerin 2 (MMRN2), shock protein 70-1A (HSP70-1A), a epidermal growth factor (EGF), EGFR, or any combination thereof. In some aspects, the anti-angiogenesis agent comprises bevacizumab (also known as AVASTIN®), ramucirumab (also known as CYRAMZA®), aflibercept (also known as EYLEA® or ZALTRAP®), tanibirumab, olaratumab (also known as LARTRUVO™), nesvacumab, AMG780, MEDI3617, vanucizumab, rilotumumab, ficlatuzumab, TAK-701, onartuzumab, emibetuzumab, or any combination thereof. In some aspects, the anti-angiogenesis agent is bevacizumab.

In some aspects, the checkpoint stimulator comprises an agonist of B7-1, B7-2, CD28, 4-1BB (CD137), 4-1BBL, GITR, inducible T cell co-stimulator (ICOS), ICOS-L, OX40, OX40L, CD70, CD27, CD40, death receptor 3 (DR3), CD28H, or any combination thereof.

In some aspects, the chemotherapeutic agent comprises an alkylating agent, an antimetabolite, an antineoplastic antibiotic, a mitotic inhibitor, a hormone or hormone modulator, a protein tyrosine kinase inhibitor, an epidermal growth factor inhibitor, a proteasome inhibitor, other neoplastic agent, or any combination thereof.

In some aspects, the immunotherapeutic agent comprises an antibody that specifically binds to EGFR (e.g., cetuximab (ERBITUX®)), ALK, ROS-1, NTRK, BRAF, ICOS, CD137 (4-1BB), CD134 (OX40), NKG2A, CD27, CD96, GITR, Herpes Virus Entry Mediator (HVEM), PD-1, PD-L1, CTLA-4, BTLA, TIM-3, A2aR, Killer cell Lectin-like Receptor G1 (KLRG-1), Natural Killer Cell Receptor 2B4 (CD244), CD160, TIGIT, VISTA, KIR, TGFβ, IL-10, IL-8, B7-H4, Fas ligand, CSF1R, CXCR4, mesothelin, CEACAM-1, CD52, HER2, MICA, MICB, or any combination thereof.

In some aspects, the platinum agent comprises cisplatin, carboplatin, oxaliplatin, satraplatin, picoplatin, nedaplatin, triplatin (e.g., triplatin tetranitrate), lipoplatin, phenanthriplatin, or any combination thereof.

In some aspects, the alkylating agent comprises altretamine, bendamustine, busulfan, carboplatin, carmustine, chlorambucil, cisplatin, cyclophosphamide, dacarbazine, ifosfamide, lomustine, mechlorethamine, melphalan, oxaliplatin, procarbazine, streptozocin, temozolomide, thiotepa, or any combination thereof.

In some aspects, the taxane comprises paclitaxel, albumin-bound paclitaxel (i.e., nab-paclitaxel), docetaxel, cabazitaxel, or any combination thereof.

In some aspects, the nucleoside analog comprises cytarabine, gemcitabine, lamivudine, entecavir, telbivudine, or any combination thereof.

In some aspects, the antimetabolite comprises capecitabine, cladribine, clofarabine, cytarabine, floxuridine, fludarabine, fluorouracil, mercaptopurine, methotrexate, pemetrexed, pentostatin, pralatrexate, thioguanine, or any combination thereof.

In some embodiments, the topoisomerase inhibitor comprises etoposide, mitoxantrone, doxorubicin, irinotecan, topotecan, camptothecin, or any combination thereof.

In some aspects, the anthracycline is doxorubicin, daunorubicin, epirubicin, idarubicin, or any combination thereof.

In some aspects, the vinca alkaloid is vinblastine, vincristine, vinorelbine, vindesine, vincaminol, vineridine, vinburnine, or any combination thereof.

### II.D.1. Checkpoint Inhibitors

In some aspects, the anti-cancer agent that is administered as an additional therapeutic agent in the methods of the disclosure is a checkpoint inhibitor.

In some aspects, the checkpoint inhibitor comprises a programmed death-1 (PD-1) pathway inhibitor, a cytotoxic T-lymphocyte-associated protein 4 (CTLA-4) inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

In some aspects, the checkpoint inhibitor is formulated for intravenous administration.

In some aspects, the checkpoint inhibitor is formulated separately from the LAG-3 antagonist and/or the CTLA-4 inhibitor. In some aspects, each checkpoint inhibitor is formulated separately when the checkpoint inhibitor comprises more than one checkpoint inhibitor.

In some aspects, the checkpoint inhibitor is administered before the LAG-3 antagonist and/or the CTLA-4 inhibitor.

In some aspects, the LAG-3 antagonist and/or the CTLA-4 inhibitor is administered before the checkpoint inhibitor.

In some aspects, the checkpoint inhibitor is formulated together with the LAG-3 antagonist and/or the CTLA-4 inhibitor. In some aspects, two or more checkpoint inhibitors are formulated together when the checkpoint inhibitor comprises more than one checkpoint inhibitor.

In some aspects, the checkpoint inhibitor is administered concurrently with the LAG-3 antagonist and/or the CTLA-4 inhibitor.

In some aspects, the checkpoint inhibitor is administered at a flat dose.

In some aspects, the checkpoint inhibitor is administered at a dose of from at least about 0.25 mg to about 2000 mg, about 0.25 mg to about 1600 mg, about 0.25 mg to about 1200 mg, about 0.25 mg to about 800 mg, about 0.25 mg to about 400 mg, about 0.25 mg to about 100 mg, about 0.25 mg to about 50 mg, about 0.25 mg to about 40 mg, about 0.25 mg to about 30 mg, about 0.25 mg to about 20 mg, about 20 mg to about 2000 mg, about 20 mg to about 1600 mg, about 20 mg to about 1200 mg, about 20 mg to about 800 mg, about 20 mg to about 400 mg, about 20 mg to about 100 mg, about 100 mg to about 2000 mg, about 100 mg to about 1800 mg, about 100 mg to about 1600 mg, about 100 mg to about 1400 mg, about 100 mg to about 1200 mg, about 100 mg to about 1000 mg, about 100 mg to about 800 mg, about 100 mg to about 600 mg, about 100 mg to about 400 mg, about 400 mg to about 2000 mg, about 400 mg to about 1800 mg, about 400 mg to about 1600 mg, about 400 mg to about 1400 mg, about 400 mg to about 1200 mg, or about 400 mg to about 1000 mg.

In some aspects, the checkpoint inhibitor is administered at a dose of about 0.25 mg, about 0.5 mg, about 0.75 mg, about 1 mg, about 1.25 mg, about 1.5 mg, about 1.75 mg, about 2 mg, about 2.25 mg, about 2.5 mg, about 2.75 mg, about 3 mg, about 3.25 mg, about 3.5 mg, about 3.75 mg, about 4 mg, about 4.25 mg, about 4.5 mg, about 4.75 mg, about 5 mg, about 5.25 mg, about 5.5 mg, about 5.75 mg, about 6 mg, about 6.25 mg, about 6.5 mg, about 6.75 mg, about 7 mg, about 7.25 mg, about 7.5 mg, about 7.75 mg, about 8 mg, about 8.25 mg, about 8.5 mg, about 8.75 mg, about 9 mg, about 9.25 mg, about 9.5 mg, about 9.75 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, about 1000 mg, about 1040 mg, about 1080 mg, about 1100 mg, about 1140 mg, about 1180 mg, about 1200 mg, about 1240 mg, about 1280 mg, about 1300 mg, about 1340 mg, about 1380 mg, about 1400 mg, about 1440 mg, about 1480 mg, about 1500 mg, about 1540 mg, about 1580 mg, about 1600 mg, about 1640 mg, about 1680 mg, about 1700 mg, about 1740 mg, about 1780 mg, about 1800 mg, about 1840 mg, about 1880 mg, about 1900 mg, about 1940 mg, about 1980 mg, or about 2000 mg.

In some aspects, the checkpoint inhibitor is administered as a weight-based dose.

In some aspects, the checkpoint inhibitor is administered at a dose from about 0.003 mg/kg to about 25 mg/kg, about 0.003 mg/kg to about 20 mg/kg, about 0.003 mg/kg to about 15 mg/kg, about 0.003 mg/kg to about 10 mg/kg, about 0.003 mg/kg to about 5 mg/kg, about 0.003 mg/kg to about 1 mg/kg, about 0.003 mg/kg to about 0.9 mg/kg, about 0.003 mg/kg to about 0.8 mg/kg, about 0.003 mg/kg to about 0.7 mg/kg, about 0.003 mg/kg to about 0.6 mg/kg, about 0.003 mg/kg to about 0.5 mg/kg, about 0.003 mg/kg to about 0.4 mg/kg, about 0.003 mg/kg to about 0.3 mg/kg, about 0.003 mg/kg to about 0.2 mg/kg, about 0.003 mg/kg to about 0.1 mg/kg, about 0.1 mg/kg to about 25 mg/kg, about 0.1 mg/kg to about 20 mg/kg, about 0.1 mg/kg to about 15 mg/kg, about 0.1 mg/kg to about 10 mg/kg, about 0.1 mg/kg to about 5 mg/kg, about 0.1 mg/kg to about 1 mg/kg, about 1 mg/kg to about 25 mg/kg, about 1 mg/kg to about 20 mg/kg, about 1 mg/kg to about 15 mg/kg, about 1 mg/kg to about 10 mg/kg, about 1 mg/kg to about 5 mg/kg, about 5 mg/kg to about 25 mg/kg, about 5 mg/kg to about 20 mg/kg, about 5 mg/kg to about 15 mg/kg, about 5 mg/kg to about 10 mg/kg, about 10 mg/kg to about 25 mg/kg, about 10 mg/kg to about 20 mg/kg, about 10 mg/kg to about 15 mg/kg, about 15 mg/kg to about 25 mg/kg, about 15 mg/kg to about 20 mg/kg, or about 20 mg/kg to about 25 mg/kg.

In some aspects, the checkpoint inhibitor is administered at a dose of about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg, about 10.0 mg/kg, about 11.0 mg/kg, about 12.0 mg/kg, about 13.0 mg/kg, about 14.0 mg/kg, about 15.0 mg/kg, about 16.0 mg/kg, about 17.0 mg/kg, about 18.0 mg/kg, about 19.0 mg/kg, about 20.0 mg/kg, about 21.0 mg/kg, about 22.0 mg/kg, about 23.0 mg/kg, about 24.0 mg/kg, or about 25.0 mg/kg.

In some aspects, the dose of the checkpoint inhibitor is administered every one week, every two weeks, every three weeks, every four weeks, every five weeks, every six weeks, every seven weeks, every eight weeks, every nine weeks, every ten weeks, every eleven weeks, or every twelve weeks.

In some aspects, each dose of the LAG-3 antagonist, the CTLA-4 inhibitor, and/or the checkpoint inhibitor as disclosed herein is administered in a constant amount.

In some aspects, each dose of the LAG-3 antagonist, the CTLA-4 inhibitor, and/or the checkpoint inhibitor as disclosed herein is administered in a varying amount. For example, in some aspects, the maintenance (or follow-on) dose of the LAG-3 antagonist, the CTLA-4 inhibitor, and/or the checkpoint inhibitor can be higher or the same as the loading dose that is first administered to the patient. In some aspects, the maintenance dose of the LAG-3 antagonist, the CTLA-4 inhibitor, and/or the checkpoint inhibitor can be lower or the same as the loading dose.

II.D.1.a. PD-1 Pathway Inhibitors

In some aspects, the checkpoint inhibitor for use in the methods of the disclosure comprises a PD-1 pathway inhibitor.

In some aspects the PD-1 pathway inhibitor is a PD-1 inhibitor and/or a PD-L1 inhibitor.

In some aspects, the PD-1 inhibitor and/or PD-L1 inhibitor is a small molecule.

In some aspects, the PD-1 inhibitor and/or PD-L1 inhibitor is a millamolecule.

In some aspects, the PD-1 inhibitor and/or PD-L1 inhibitor is a macrocyclic peptide.

In certain aspects, the PD-1 inhibitor and/or PD-L1 inhibitor is BMS-986189.

In some aspects, the PD-1 inhibitor is an inhibitor disclosed in International Publication No. WO2014/151634, which is incorporated by reference herein in its entirety.

In some aspects, the PD-1 inhibitor is INCMGA00012 (Insight Pharmaceuticals).

In some aspects, the PD-1 inhibitor comprises a combination of an anti-PD-1 antibody disclosed herein and a PD-1 small molecule inhibitor.

In some aspects, the PD-L1 inhibitor comprises a millamolecule having a formula set forth in formula (I):

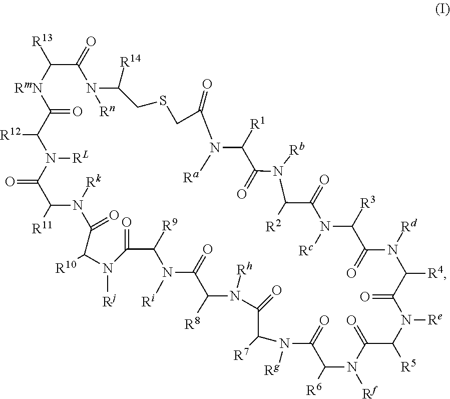

wherein R¹-R¹³ are amino acid side chains, R^{a}-Rⁿ are hydrogen, methyl, or form a ring with a vicinal R group, and R¹⁴ is —C(O)NHR¹¹, wherein R¹⁵ is hydrogen, or a glycine residue optionally substituted with additional glycine residues and/or tails which can improve pharmacokinetic properties. In some aspects, the PD-L1 inhibitor comprises a compound disclosed in International Publication No. WO2014/151634, which is incorporated by reference herein in its entirety. In some aspects, the PD-L1 inhibitor comprises a compound disclosed in International Publication No. WO2016/039749, WO2016/149351, WO2016/077518, WO2016/100285, WO2016/100608, WO2016/126646, WO2016/057624, WO2017/151830, WO2017/176608, WO2018/085750, WO2018/237153, or WO2019/070643, each of which is incorporated by reference herein in its entirety.

In some aspects, the PD-L1 inhibitor comprises a small molecule PD-L1 inhibitor disclosed in International Publication No. WO2015/034820, WO2015/160641, WO2018/044963, WO2017/066227, WO2018/009505, WO2018/183171, WO2018/118848, WO2019/147662, or WO2019/169123, each of which is incorporated by reference herein in its entirety.

In some aspects, the PD-1 pathway inhibitor is a soluble PD-L2 polypeptide. In some aspects, the soluble PD-L2 polypeptide is a fusion polypeptide. In some aspects, the soluble PD-L2 polypeptide comprises a ligand binding fragment of the PD-L2 extracellular domain. In some aspects, the soluble PD-L2 polypeptide further comprises a half-life extending moiety. In some aspects, the half-life extending moiety comprises an immunoglobulin constant region or a portion thereof, an immunoglobulin-binding polypeptide, an immunoglobulin G (IgG), albumin-binding polypeptide (ABP), a PASylation moiety, a HESylation moiety, XTEN, a PEGylation moiety, an Fc region, or any combination thereof. In some aspects, the soluble PD-L2 polypeptide is AMP-224 (see, e.g., US 2013/0017199).

In some aspects, the PD-1 pathway inhibitor is an anti-PD-1 antibody and/or an anti-PD-L1 antibody.

II.D.1.a.i. Anti-PD-1 Antibodies

Anti-PD-1 antibodies that are known in the art can be used in the methods of the disclosure. Various human monoclonal antibodies that bind specifically to PD-1 with high affinity have been disclosed in U.S. Pat. No. 8,008,449. Anti-PD-1 human antibodies disclosed in U.S. Pat. No. 8,008,449 have been demonstrated to exhibit one or more of the following characteristics: (a) bind to human PD-1 with a K_{D} of 1×10⁻⁷ M or less, as determined by surface plasmon resonance using a Biacore biosensor system; (b) do not substantially bind to human CD28, CTLA-4 or ICOS; (c) increase T-cell proliferation in a Mixed Lymphocyte Reaction (MLR) assay; (d) increase interferon-7 production in an MLR assay; (e) increase IL-2 secretion in an MLR assay; (f) bind to human PD-1 and cynomolgus monkey PD-1; (g) inhibit the binding of PD-L1 and/or PD-L2 to PD-1; (h) stimulate antigen-specific memory responses; (i) stimulate antibody responses; and (j) inhibit tumor cell growth in vivo. Anti-PD-1 antibodies usable in the present disclosure include monoclonal antibodies that bind specifically to human PD-1 and exhibit at least one, in some aspects, at least five, of the preceding characteristics.

Other anti-PD-1 monoclonal antibodies that can be used in the methods of the disclosure have been described in, for example, U.S. Pat. Nos. 6,808,710, 7,488,802, 8,168,757 and 8,354,509, US Publication No. 2016/0272708, and PCT Publication Nos. WO 2012/145493, WO 2008/156712, WO 2015/112900, WO 2012/145493, WO 2015/112800, WO 2014/206107, WO 2015/35606, WO 2015/085847, WO 2014/179664, WO 2017/020291, WO 2017/020858, WO 2016/197367, WO 2017/024515, WO 2017/025051, WO 2017/123557, WO 2016/106159, WO 2014/194302, WO 2017/040790, WO 2017/133540, WO 2017/132827, WO 2017/024465, WO 2017/025016, WO 2017/106061, WO 2017/19846, WO 2017/024465, WO 2017/025016, WO 2017/132825, and WO 2017/133540 each of which is incorporated by reference in its entirety.

Anti-PD-1 antibodies that can be used in the methods of the disclosure include nivolumab (also known as OPDIVO®, 5C4, BMS-936558, MDX-1106, and ONO-4538), pembrolizumab (Merck; also known as KEYTRUDA®, lambrolizumab, and MK3475; see WO 2008/156712), PDR001 (Novartis; also known as spartalizumab; see WO 2015/112900 and U.S. Pat. No. 9,683,048), MEDI-0680 (AstraZeneca; also known as AMP-514; see WO 2012/145493), TSR-042 (Tesaro Biopharmaceutical; also known as ANBO11 or dostarlimab; see WO 2014/179664), cemiplimab (Regeneron; also known as LIBTAYO® or REGN2810; see WO 2015/112800 and U.S. Pat. No. 9,987,500), JS001 (TAIZHOU JUNSHI PHARMA; also known as toripalimab; see Si-Yang Liu et al., J. Hematol. Oncol. 10:136 (2017)), PF-06801591 (Pfizer; also known as sasanlimab; US 2016/0159905), BGB-A317 (Beigene; also known as tislelizumab; see WO 2015/35606 and US 2015/0079109), BI 754091 (Boehringer Ingelheim; see Zettl M et al., Cancer. Res. (2018); 78(13 Suppl):Abstract 4558), INCSHR1210 (Jiangsu Hengrui Medicine; also known as SHR-1210 or camrelizumab; see WO 2015/085847; Si-Yang Liu et al., J. Hematol. Oncol. 10:136 (2017)), GLS-010 (Wuxi/Harbin Gloria Pharmaceuticals; also known as WBP3055; see Si-Yang Liu et al., J Hematol. Oncol. 10:136 (2017)), AM-0001 (Armo), STI-1110 (Sorrento Therapeutics; see WO 2014/194302), AGEN2034 (Agenus; see WO 2017/040790), MGA012 (Macrogenics, see WO 2017/19846), BCD-100 (Biocad; Kaplon et al., mAbs 10(2):183-203 (2018), IBI308 (Innovent; also known as sintilimab; see WO 2017/024465, WO 2017/025016, WO 2017/132825, and WO 2017/133540), and SSI-361 (Lyvgen Biopharma Holdings Limited, US 2018/0346569).

In some aspects, the anti-PD-1 antibody comprises sequences of the heavy and light chain CDRs, heavy and light chain variable regions, or heavy and light chains of an anti-PD-1 antibody disclosed herein or as known in the art, such as sequences provided in the publications disclosed herein.

In some aspects, a method as disclosed herein comprises an anti-PD-1 antibody having at least about 90% sequence identity with an anti-PD-1 antibody as disclosed herein or as known in the art (e.g., at least about 90%, about 91%, about 92%, about 93%, about 94%, about 95%, about 96%, about 97%, about 98%, or about 99% identity to a sequence of an anti-PD-1 antibody, such as to the heavy chain variable region and/or light chain variable region or to the heavy chain and/or light chain of an anti-PD-1 antibody).

Anti-PD-1 antibodies that can be used in the methods of the disclosure also include isolated antibodies that bind specifically to human PD-1 and cross-compete for binding to human PD-1 with any anti-PD-1 antibody disclosed herein, e.g., nivolumab (see, e.g., U.S. Pat. Nos. 8,008,449 and 8,779,105; WO 2013/173223). In some aspects, the anti-PD-1 antibody binds the same epitope as any of the anti-PD-1 antibodies described herein, e.g., nivolumab.

In some aspects, the antibodies that cross-compete for binding to human PD-1 with, or bind to the same epitope region as, any anti-PD-1 antibody disclosed herein, e.g., nivolumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-1 antibodies that can be used in the methods of the disclosure also include antigen-binding portions of any of the above full-length antibodies.

Anti-PD-1 antibodies that can be used in the methods of the disclosure are antibodies that bind to PD-1 with high specificity and affinity, block the binding of PD-L1 and or PD-L2, and inhibit the immunosuppressive effect of the PD-1 signaling pathway. In any of the compositions or methods disclosed herein, an anti-PD-1 “antibody” includes an antigen-binding portion or fragment that binds to the PD-1 receptor and exhibits the functional properties similar to those of whole antibodies in inhibiting ligand binding and up-regulating the immune system. In certain aspects, the anti-PD-1 antibody or antigen-binding portion thereof cross-competes with nivolumab for binding to human PD-1.

In some aspects, the anti-PD-1 antibody is a full-length antibody. In some aspects, the anti-PD-1 antibody is a monoclonal, human, humanized, chimeric, or multispecific antibody. In some aspects, the multispecific antibody is a DART, a DVD-Ig, or bispecific antibody.

In some aspects, the anti-PD-1 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-PD-1 antibody is nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or comprises an antigen binding portion thereof.

In some aspects, the anti-PD-1 antibody is formulated for intravenous administration.

In some aspects, the anti-PD-1 antibody is administered intravenously for about 30 minutes.

In some aspects, the anti-PD-1 antibody is nivolumab. Nivolumab is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

In some aspects, nivolumab is administered at a dose of about 240 mg once about every 2 weeks.

In some aspects, nivolumab is administered at a dose of about 480 mg once about every 4 weeks.

In some aspects, nivolumab is administered at a dose of about 1 mg/kg, followed by ipilimumab on the same day, once about every 3 weeks for about 4 doses, then nivolumab at about 240 mg once about every 2 weeks or at about 480 mg once about every 4 weeks.

In some aspects, the anti-PD-1 antibody is pembrolizumab. Pembrolizumab is a humanized monoclonal IgG4 (S228P) antibody directed against human cell surface receptor PD-1. Pembrolizumab is described, for example, in U.S. Pat. Nos. 8,354,509 and 8,900,587.

In some aspects, pembrolizumab is administered at a dose of about 200 mg once about every 2 weeks. In some aspects, pembrolizumab is administered at a dose of about 200 mg once about every 3 weeks. In some aspects, pembrolizumab is administered at a dose of about 400 mg once about every 6 weeks. In some aspects, pembrolizumab is administered at a dose of about 300 mg once about every 4-5 weeks.

In some aspects, the anti-PD-1 antibody is cemiplimab (REGN2810). Cemiplimab is described, for example, in WO 2015/112800 and U.S. Pat. No. 9,987,500.

In some aspects, cemiplimab is administered intravenously at a dose of about 3 mg/kg or about 350 mg once about every 3 weeks.

In some aspects, the anti-PD-1 antibody is spartalizumab (PDR001). Spartalizumab is described, for example, in WO 2015/112900 and U.S. Pat. No. 9,683,048.

In some aspects, spartalizumab is administered intravenously at a dose of about 300 mg once about every 3 weeks or 400 mg once about every 4 weeks.

II.D.1.a.ii. Anti-PD-L1 Antibodies

Anti-PD-L1 antibodies that are known in the art can be used in the methods of the disclosure. Examples of anti-PD-L1 antibodies useful in the compositions and methods of the present disclosure include the antibodies disclosed in U.S. Pat. No. 9,580,507. Anti-PD-L1 human monoclonal antibodies disclosed in U.S. Pat. No. 9,580,507 have been demonstrated to exhibit one or more of the following characteristics: (a) bind to human PD-L1 with a K_{D} of 1×10⁻⁷ M or less, as determined by surface plasmon resonance using a Biacore biosensor system; (b) increase T-cell proliferation in a Mixed Lymphocyte Reaction (MLR) assay; (c) increase interferon-7 production in an MLR assay; (d) increase IL-2 secretion in an MLR assay; (e) stimulate antibody responses; and (f) reverse the effect of T regulatory cells on T cell effector cells and/or dendritic cells. Anti-PD-L1 antibodies usable in the present disclosure include monoclonal antibodies that bind specifically to human PD-L1 and exhibit at least one, in some aspects, at least five, of the preceding characteristics.

Anti-PD-L1 antibodies that can be used in the methods of the disclosure include BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 31(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), BGB-A333 (BeiGene; see Desai et al., JCO 36 (15suppl):TPS3113 (2018)), ICO 36, and CK-301 (Checkpoint Therapeutics; see Gorelik et al., AACR:Abstract 4606 (April 2016)).

In some aspects, the anti-PD-L1 antibody comprises sequences of the heavy and light chain CDRs, heavy and light chain variable regions, or heavy and light chains of an anti-PD-L1 antibody disclosed herein or as known in the art, such as sequences provided in the publications disclosed herein.

In some aspects, a method as disclosed herein comprises an anti-PD-L1 antibody having at least about 90% sequence identity with an anti-PD-L1 antibody as disclosed herein or as known in the art (e.g., at least about 90%, about 91%, about 92%, about 93%, about 94%, about 95%, about 96%, about 97%, about 98%, or about 99% identity to a sequence of an anti-PD-L1 antibody, such as to the heavy chain variable region and/or light chain variable region or to the heavy chain and/or light chain of an anti-PD-L1 antibody).

Anti-PD-L1 antibodies that can be used in the methods of the disclosure also include isolated antibodies that bind specifically to human PD-L1 and cross-compete for binding to human PD-L1 with any anti-PD-L1 antibody disclosed herein, e.g., atezolizumab, durvalumab, and/or avelumab. In some aspects, the anti-PD-L1 antibody binds the same epitope as any of the anti-PD-L1 antibodies described herein, e.g., atezolizumab, durvalumab, and/or avelumab. In certain aspects, the antibodies that cross-compete for binding to human PD-L1 with, or bind to the same epitope region as, any anti-PD-L1 antibody disclosed herein, e.g., atezolizumab, durvalumab, and/or avelumab, are monoclonal antibodies. For administration to human subjects, these cross-competing antibodies are chimeric antibodies, engineered antibodies, or humanized or human antibodies. Such chimeric, engineered, humanized or human monoclonal antibodies can be prepared and isolated by methods well known in the art.

Anti-PD-L1 antibodies that can be used in the methods of the disclosure also include antigen-binding portions of any of the above full-length antibodies.

Anti-PD-L1 antibodies that can be used in the methods of the disclosure are antibodies that bind to PD-L1 with high specificity and affinity, block the binding of PD-1, and inhibit the immunosuppressive effect of the PD-1 signaling pathway. In any of the compositions or methods disclosed herein, an anti-PD-L1 “antibody” includes an antigen-binding portion or fragment that binds to PD-L1 and exhibits the functional properties similar to those of whole antibodies in inhibiting receptor binding and up-regulating the immune system. In certain aspects, the anti-PD-L1 antibody or antigen-binding portion thereof cross-competes with atezolizumab, durvalumab, and/or avelumab for binding to human PD-L1.

In some aspects, an anti-PD-L1 antibody is substituted for the anti-PD-1 antibody in any of the methods disclosed herein.

In some aspects, the anti-PD-L1 antibody is a full-length antibody.

In some aspects, the anti-PD-L1 antibody is a monoclonal, human, humanized, chimeric, or multispecific antibody. In some aspects, the multispecific antibody is a DART, a DVD-Ig, or bispecific antibody.

In some aspects, the anti-PD-L1 antibody is a F(ab′)2 fragment, a Fab′ fragment, a Fab fragment, a Fv fragment, a scFv fragment, a dsFv fragment, a dAb fragment, or a single chain binding polypeptide.

In some aspects, the anti-PD-L1 antibody is BMS-936559, atezolizumab, durvalumab, avelumab, STI-1014, CX-072, KN035, LY3300054, BGB-A333, ICO 36, CK-301, or comprises an antigen binding portion thereof.

In some aspects, the PD-L1 antibody is atezolizumab. Atezolizumab is a fully humanized IgG1 monoclonal anti-PD-L1 antibody. In some aspects, atezolizumab is administered as a flat dose of about 800 mg once about every 2 weeks. In some aspects, atezolizumab is administered as a flat dose of about 840 mg once about every 2 weeks.

In some aspects, the PD-L1 antibody is durvalumab. Durvalumab is a human IgG1 kappa monoclonal anti-PD-L1 antibody. In some aspects, durvalumab is administered at a dose of about 10 mg/kg once about every 2 weeks. In some aspects, durvalumab is administered at a dose of about 10 mg/kg once about every 2 weeks for up to 12 months. In some aspects, durvalumab is administered as a flat dose of about 800 mg/kg once about every 2 weeks. In some aspects, durvalumab is administered as a flat dose of about 1200 mg/kg once about every 3 weeks.

In some aspects, the PD-L1 antibody is avelumab. Avelumab is a human IgG1 lambda monoclonal anti-PD-L1 antibody. In some aspects, avelumab is administered as a flat dose of about 800 mg once about every 2 weeks.

### III. Pharmaceutical Compositions

LAG-3 antagonists (e.g., anti-LAG-3 antibodies), CTLA-4 inhibitors (e.g., anti-CTLA-4 antibodies), PD-1 pathway inhibitors (e.g., anti-PD-1 antibodies), and/or other therapeutic agents of the present disclosure can be constituted in a composition, e.g., one or more pharmaceutical compositions containing a LAG-3 antagonist, CTLA-4 inhibitor, PD-1 pathway inhibitor, and/or other therapeutic agent as disclosed herein and a pharmaceutically acceptable carrier. The LAG-3 antagonist, CTLA-4 inhibitor, PD-1 pathway inhibitor, and/or other therapeutic agent can be formulated together or separately in any combination. As used herein, a “pharmaceutically acceptable carrier” includes any and all solvents, dispersion media, coatings, antibacterial and antifungal agents, isotonic and absorption delaying agents, and the like that are physiologically compatible.

In some aspects, the carrier for a composition containing a LAG-3 antagonist, CTLA-4 inhibitor, PD-1 pathway inhibitor, and/or other therapeutic agent as disclosed herein is suitable for intravenous, intramuscular, subcutaneous, parenteral, spinal or epidermal administration (e.g., by injection or infusion). In some aspects, the carrier is suitable for non-parenteral, e.g., oral, administration. In some aspects, a subcutaneous injection is based on Halozyme Therapeutics' ENHANZE® drug-delivery technology (see U.S. Pat. No. 7,767,429, which is incorporated by reference herein in its entirety). ENHANZE® uses a co-formulation of an antibody with recombinant human hyaluronidase enzyme (rHuPH20), which removes traditional limitations on the volume of biologics and drugs that can be delivered subcutaneously due to the extracellular matrix (see U.S. Pat. No. 7,767,429). A pharmaceutical composition of the disclosure can include one or more pharmaceutically acceptable salts, anti-oxidant, aqueous and non-aqueous carriers, and/or adjuvants such as preservatives, wetting agents, emulsifying agents and dispersing agents. In some aspects, the pharmaceutical composition for the present disclosure can further comprise recombinant human hyaluronidase enzyme, e.g., rHuPH20.

Treatment is continued as long as clinical benefit is observed or until unacceptable toxicity or disease progression occurs. Dosage and frequency vary depending on the half-life of the LAG-3 antagonist, CTLA-4 inhibitor, PD-1 pathway inhibitor, and/or other therapeutic agent in the patient. In general, for antibodies as disclosed herein, human antibodies show the longest half-life, followed by humanized antibodies, chimeric antibodies, and nonhuman antibodies. The dosage and frequency of administration can vary depending on whether the treatment is prophylactic or therapeutic. In prophylactic applications, a relatively low dosage is typically administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients (e.g., LAG-3 antagonist, CTLA-4 inhibitor, PD-1 pathway inhibitor, and/or other therapeutic agent) in the pharmaceutical compositions of the present disclosure can be varied so as to obtain an amount of the active ingredient which is effective to achieve the desired therapeutic response for a particular patient, composition, and mode of administration, without being unduly toxic to the patient. The selected dosage level will depend upon a variety of pharmacokinetic factors including the activity of the particular compositions of the present disclosure employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. A composition of the present disclosure can be administered via one or more routes of administration using one or more of a variety of methods well known in the art. As will be appreciated by the skilled artisan, the route and/or mode of administration will vary depending upon the desired results.

### IV. Patient Populations

Provided herein are clinical methods for treating unresectable or metastatic melanoma in a human patient comprising an immunotherapy disclosed herein, for example, a LAG-3 antagonist (e.g., an anti-LAG-3 antibody), or a combination of a LAG-3 antagonist and a CTLA-4 inhibitor (e.g., an anti-CTLA-4 antibody). In certain aspects, the patient who receives the immunotherapy disclosed herein has a sensitizing mutation for a targeted inhibitor therapy. In certain aspects, the patient who receives the immunotherapy disclosed herein has received a prior PD-1 pathway inhibitor as a treatment for melanoma. In some aspects, the prior PD-1 pathway inhibitor is an anti-PD-1 antibody. In one aspect, the patient received the prior PD-1 pathway inhibitor in a time period greater than or equal to 6 months between the last dose and the date of recurrence. In another aspect, the patient suffers from unresectable or metastatic melanoma that is refractory to treatment with chemotherapy.

In an aspect, the patient suffers from unresectable or metastatic melanoma that is refractory to treatment with an immune checkpoint inhibitor. In another aspect, the patient suffers from unresectable or metastatic melanoma that is refractory to treatment with a PD-1 inhibitor. In another aspect, the patient suffers from unresectable or metastatic melanoma that is refractory to treatment with an anti-PD-1 antibody. In one aspect, the patient suffers from unresectable or metastatic melanoma that is predicted to be refractory to treatment with an anti-PD-1 antibody. In one aspect, the unresectable or metastatic melanoma is deemed refractory to treatment with an anti-PD-1 antibody based on biomarker analysis. In certain aspects, the unresectable or metastatic melanoma is refractory to monotherapy with an anti-PD-1 antibody. In another aspect, the patient suffers from unresectable metastatic or melanoma that is refractory to treatment with an anti-PD-L1 antibody.

Patients can be tested or selected for one or more of the above described clinical attributes prior to, during, or after treatment.

### V. Immunotherapies

In one aspect, immunotherapies provided herein comprise administration of a LAG-3 antagonist (e.g., an anti-LAG-3 antibody) or a combination of a LAG-3 antagonist and a CTLA-4 inhibitor (e.g., an anti-CTLA-4 antibody) to treat unresectable or metastatic melanoma.

In one aspect, the invention provides an anti-LAG-3 antibody and an anti-CTLA-4 antibody according to a defined clinical dosage regimen, to treat patients having metastatic or unresectable melanoma. In a particular aspect, the anti-LAG-3 antibody is BMS-986016 (relatlimab). In another aspect, the anti-CTLA-4 antibody is ipilimumab. In some aspects, the patient is further administered an anti-PD-1 antibody. In further aspects, the patient is additionally administered chemotherapy.

As used herein, adjunctive or combined administration (co-administration) includes simultaneous administration of the compounds in the same or different dosage form, or separate administration of the compounds (e.g., sequential administration). Thus, for example, the anti-LAG-3 and anti-CTLA-4 antibodies can be simultaneously administered in a single formulation. Alternatively, the anti-LAG-3 and anti-CTLA-4 antibodies can be formulated for separate administration and are administered concurrently or sequentially (e.g., one antibody is administered within about 30 minutes prior to administration of the second antibody).

For example, the anti-CTLA-4 antibodies antibody can be administered first, followed by (e.g., immediately followed by) the administration of the anti-LAG-3 antibody, or vice versa. In one aspect, the anti-CTLA-4 antibody is administered prior to administration of the anti-LAG-3 antibody. In another aspect, the anti-CTLA-4 antibody is administered after administration of the anti-LAG-3 antibody. In another aspect, the anti-LAG-3 antibody and anti-CTLA-4 antibody are administered concurrently. Such concurrent or sequential administration preferably results in both antibodies being simultaneously present in treated patients.

### VI. Treatment Protocols

Suitable treatment protocols for the methods of the disclosure comprise administering to the patient an effective amount of a LAG-3 antagonist (e.g., an anti-LAG-3 antibody) or an effective amount of a LAG-3 antagonist and an effective amount of a CTLA-4 inhibitor (e.g., an anti-CTLA-4 antibody).

In some aspects, a suitable protocol comprises, for example, administering to the patient an effective amount of any anti-LAG-3 antibody of the disclosure in combination with an effective amount of any anti-CTLA-4 antibody of the disclosure.

In some aspects, a suitable treatment protocol comprises, for example, administering to the patient an effective amount of each of: (a) an anti-LAG-3 antibody, such as one comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) an anti-CTLA-4 antibody, such as one comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

In some aspects, a suitable treatment protocol comprises any of the doses of the disclosure for a LAG-3 antagonist and/or a CTLA-4 inhibitor.

In some aspects, a suitable treatment protocol comprises any duration of administration (e.g., any administration cycle) of the disclosure.

In some aspects, a suitable treatment protocol comprises a dose of about 360 mg of an anti-LAG-3 antibody and about 3 mg/kg of an anti-CTLA-4 antibody.

In some aspects, a suitable treatment protocol comprises a dose of about 720 mg of an anti-LAG-3 antibody and about 3 mg/kg of an anti-CTLA-4 antibody.

In some aspects, a suitable treatment protocol comprises a dose of about 1080 mg of an anti-LAG-3 antibody and about 3 mg/kg of an anti-CTLA-4 antibody.

In some aspects, a suitable treatment protocol comprises a dose of about 1200 mg of an anti-LAG-3 antibody and about 3 mg/kg of an anti-CTLA-4 antibody.

In some aspects, a suitable treatment protocol comprises a patient who has received a prior PD-1 pathway inhibitor as a treatment for melanoma.

Provided herein is a method of inhibiting the growth of an unresectable or metastatic melanoma tumor in a human patient, the method comprising administering to the patient: (a) a dose of about 360 mg, about 720 mg, about 1080 mg, or about 1200 mg of an anti-LAG-3 antibody; and (b) a dose of about 3 mg/kg of an anti-CTLA-4 antibody. In some aspects, the patient has received a prior PD-1 pathway inhibitor as a treatment for melanoma. In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and the anti-CTLA-4 antibody comprises CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32. In some aspects, the anti-LAG-3 antibody is relatlimab and the anti-CTLA-4 antibody is ipilimumab. In some aspects, a cycle of administration is three weeks (Q3W), which can be repeated, as necessary.

Provided herein is a method of treating an unresectable or metastatic melanoma in a human patient, the method comprising administering to the patient: (a) a dose of about 360 mg, about 720 mg, about 1080 mg, or about 1200 mg of anti-LAG-3 antibody; and (b) a dose of about 3 mg/kg of an anti-CTLA-4 antibody. In some aspects, the patient has received a prior PD-1 pathway inhibitor as a treatment for melanoma. In some aspects, the anti-LAG-3 antibody comprises CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and the anti-CTLA-4 antibody comprises CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32. In some aspects, the anti-LAG-3 antibody is relatlimab and the anti-CTLA-4 antibody is ipilimumab. In some aspects, a cycle of administration is three weeks (Q3W), which can be repeated, as necessary.

Provided herein is a method of treating unresectable or metastatic melanoma in a human patient, the method comprising administering to the patient: (a) a dose of about 360 mg of an anti-LAG-3 antibody, and (b) a dose of about 3 mg/kg of an anti-CTLA-4 antibody.

Provided herein is a method of treating unresectable or metastatic melanoma in a human patient, the method comprising administering to the patient: (a) a dose of about 720 mg of an anti-LAG-3 antibody, and (b) a dose of about 3 mg/kg of an anti-CTLA-4 antibody.

Provided herein is a method of treating unresectable or metastatic melanoma in a human patient, the method comprising administering to the patient: (a) a dose of about 1080 mg of an anti-LAG-3 antibody, and (b) a dose of about 3 mg/kg of an anti-CTLA-4 antibody.

Provided herein is a method of treating unresectable or metastatic melanoma in a human patient, the method comprising administering to the patient: (a) a dose of about 1200 mg of an anti-LAG-3 antibody, and (b) a dose of about 3 mg/kg of an anti-CTLA-4 antibody.

Provided herein is a method of treating unresectable or metastatic melanoma in a human patient, the method comprising administering to the patient: (a) a dose of about 360 mg of an anti-LAG-3 antibody, and (b) a dose of about 3 mg/kg of an anti-CTLA-4 antibody; wherein the patient has received a prior PD-1 pathway inhibitor as a treatment for melanoma.

Provided herein is a method of treating unresectable or metastatic melanoma in a human patient, the method comprising administering to the patient: (a) a dose of about 720 mg of an anti-LAG-3 antibody, and (b) a dose of about 3 mg/kg of an anti-CTLA-4 antibody; wherein the patient has received a prior PD-1 pathway inhibitor as a treatment for melanoma.

Provided herein is a method of treating unresectable or metastatic melanoma in a human patient, the method comprising administering to the patient: (a) a dose of about 1080 mg of an anti-LAG-3 antibody, and (b) a dose of about 3 mg/kg of an anti-CTLA-4 antibody; wherein the patient has received a prior PD-1 pathway inhibitor as a treatment for melanoma.

Provided herein is a method of treating unresectable or metastatic melanoma in a human patient, the method comprising administering to the patient: (a) a dose of about 1200 mg of an anti-LAG-3 antibody, and (b) a dose of about 3 mg/kg of an anti-CTLA-4 antibody; wherein the patient has received a prior PD-1 pathway inhibitor as a treatment for melanoma.

Provided herein is a method of treating unresectable or metastatic melanoma in a human patient, the method comprising administering to the patient: (a) an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

Provided herein is a method of treating unresectable or metastatic melanoma in a human patient, the method comprising administering to the patient: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

Provided herein is a method of treating unresectable or metastatic melanoma in a human patient, the method comprising administering to the patient: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

Provided herein is a method of treating unresectable or metastatic melanoma in a human patient, the method comprising administering to the patient: (a) a dose of about 1080 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

Provided herein is a method of treating unresectable or metastatic melanoma in a human patient, the method comprising administering to the patient: (a) a dose of about 1200 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32.

Provided herein is a method of treating unresectable or metastatic melanoma in a human patient, the method comprising administering to the patient: (a) a dose of about 360 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

Provided herein is a method of treating unresectable or metastatic melanoma in a human patient, the method comprising administering to the patient: (a) a dose of about 720 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

Provided herein is a method of treating unresectable or metastatic melanoma in a human patient, the method comprising administering to the patient: (a) a dose of about 1080 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

Provided herein is a method of treating unresectable or metastatic melanoma in a human patient, the method comprising administering to the patient: (a) a dose of about 1200 mg of an anti-LAG-3 antibody comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5, and (b) a dose of about 3 mg/kg of an anti-CTLA-4 antibody comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; wherein the patient has received a prior PD-1 pathway inhibitor as a treatment for melanoma.

Provided herein is a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising: (i) a heavy chain variable region CDR1 comprising the sequence set forth in SEQ ID NO:7; (ii) a heavy chain variable region CDR2 comprising the sequence set forth in SEQ ID NO:8; (iii) a heavy chain variable region CDR3 comprising the sequence set forth in SEQ ID NO:9; (iv) a light chain variable region CDR1 comprising the sequence set forth in SEQ ID NO:10; (v) a light chain variable region CDR2 comprising the sequence set forth in SEQ ID NO: 11; and (vi) a light chain variable region CDR3 comprising the sequence set forth in SEQ ID NO:12, and (b) a dose of about 3 mg/kg of an anti-CTLA-4 antibody comprising: (i) a heavy chain variable region CDR1 comprising the sequence set forth in SEQ ID NO:35; (ii) a heavy chain variable region CDR2 comprising the sequence set forth in SEQ ID NO:36; (iii) a heavy chain variable region CDR3 comprising the sequence set forth in SEQ ID NO:37; (iv) a light chain variable region CDR1 comprising the sequence set forth in SEQ ID NO:38; (v) a light chain variable region CDR2 comprising the sequence set forth in SEQ ID NO:39; and (vi) a light chain variable region CDR3 comprising the sequence set forth in SEQ ID NO:40.

Provided herein is a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising: (i) a heavy chain variable region CDR1 comprising the sequence set forth in SEQ ID NO:7; (ii) a heavy chain variable region CDR2 comprising the sequence set forth in SEQ ID NO:8; (iii) a heavy chain variable region CDR3 comprising the sequence set forth in SEQ ID NO:9; (iv) a light chain variable region CDR1 comprising the sequence set forth in SEQ ID NO:10; (v) a light chain variable region CDR2 comprising the sequence set forth in SEQ ID NO:11; and (vi) a light chain variable region CDR3 comprising the sequence set forth in SEQ ID NO:12, and (b) a dose of about 3 mg/kg of an anti-CTLA-4 antibody comprising: (i) a heavy chain variable region CDR1 comprising the sequence set forth in SEQ ID NO:35; (ii) a heavy chain variable region CDR2 comprising the sequence set forth in SEQ ID NO:36; (iii) a heavy chain variable region CDR3 comprising the sequence set forth in SEQ ID NO:37; (iv) a light chain variable region CDR1 comprising the sequence set forth in SEQ ID NO:38; (v) a light chain variable region CDR2 comprising the sequence set forth in SEQ ID NO:39; and (vi) a light chain variable region CDR3 comprising the sequence set forth in SEQ ID NO:40; wherein the patient has received a prior PD-1 pathway inhibitor as a treatment for melanoma.

Provided herein is a method of treating unresectable or metastatic melanoma in a human patient, the method comprising administering to the patient an effective amount of each of: (a) a dose of about 360 mg, about 720 mg, about 1080 mg, or about 1200 mg of an anti-LAG-3 antibody comprising heavy and light chain variable regions comprising the sequences set forth in SEQ ID NOs:3 and 5, respectively, and (b) a dose of about 3 mg/kg of an anti-CTLA-4 antibody comprising heavy and light chain variable regions comprising the sequences set forth in SEQ ID NOs:34 and 32, respectively.

Provided herein is a method of treating unresectable or metastatic melanoma in a human patient, the method comprising administering to the patient: (a) a dose of about 360 mg, about 720 mg, about 1080 mg, or about 1200 mg of an anti-LAG-3 antibody comprising heavy and light chain variable regions comprising the sequences set forth in SEQ ID NOs:3 and 5, respectively, and (b) a dose of about 3 mg/kg of an anti-CTLA-4 antibody comprising heavy and light chain variable regions comprising the sequences set forth in SEQ ID NOs:34 and 32, respectively; wherein the patient has received a prior PD-1 pathway inhibitor as a treatment for melanoma.

In some aspects, the anti-LAG-3 antibody and the anti-CTLA-4 antibody are administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the anti-LAG-3 antibody and the anti-CTLA-4 antibody are administered once about every three weeks.

Provided herein is a method of treating unresectable or metastatic melanoma in a human patient, the method comprising administering to the patient an anti-LAG-3 antibody comprising: (a) a heavy chain variable region CDR1 comprising the sequence set forth in SEQ ID NO:7; (b) a heavy chain variable region CDR2 comprising the sequence set forth in SEQ ID NO:8; (c) a heavy chain variable region CDR3 comprising the sequence set forth in SEQ ID NO:9; (d) a light chain variable region CDR1 comprising the sequence set forth in SEQ ID NO: 10; (e) a light chain variable region CDR2 comprising the sequence set forth in SEQ ID NO: 11; and (f) a light chain variable region CDR3 comprising the sequence set forth in SEQ ID NO:12, and wherein the patient has received a prior PD-1 pathway inhibitor as a treatment for melanoma; and wherein at least one dose of the anti-LAG-3 antibody is administered at a dose of about 360 mg, about 720 mg, about 1080 mg, or about 1200 mg.

In some aspects, the anti-LAG-3 antibody is administered once about every one week, once about every two weeks, once about every three weeks, once about every four weeks, once about every five weeks, once about every six weeks, once about every seven weeks, once about every eight weeks, once about every nine weeks, once about every ten weeks, once about every eleven weeks, or once about every twelve weeks.

In some aspects, the anti-LAG-3 antibody is administered once about every three weeks.

Provided herein is a method of treating unresectable or metastatic melanoma in a human patient, the method comprising administering to the patient: (a) a dose of an anti-LAG-3 antibody, wherein the anti-LAG-3 antibody is AGEN1746, and (b) a dose of an anti-CTLA-4 antibody, wherein the anti-CTLA-4 antibody is AGEN1884, wherein the patient has received a prior PD-1 pathway inhibitor as a treatment for melanoma.

Provided herein is a method of treating unresectable or metastatic melanoma in a human patient, the method comprising administering to the patient: (a) a dose of an anti-LAG-3 antibody, wherein the anti-LAG-3 antibody is MK4280, and (b) a dose of an anti-CTLA-4 antibody, wherein the anti-CTLA-4 antibody is MK1308, wherein the patient has received a prior PD-1 pathway inhibitor as a treatment for melanoma.

Provided herein is a method of treating unresectable or metastatic melanoma in a human patient, the method comprising administering to the patient: (a) a dose of an anti-LAG-3 antibody, wherein the anti-LAG-3 antibody is REGN3767, and (b) a dose of an anti-CTLA-4 antibody, wherein the anti-CTLA-4 antibody is REGN4659, wherein the patient has received a prior PD-1 pathway inhibitor as a treatment for melanoma.

In some aspects, the anti-LAG-3 and anti-CTLA-4 antibodies are formulated for intravenous administration.

In some aspects, a cycle of administration is once about every three weeks, which can be repeated, as necessary.

In some aspects, the anti-LAG-3 antibody is BMS-986016 and the anti-CTLA-4 antibody is ipilimumab.

In some aspects, the anti-LAG-3 antibody is MK4280.

In some aspects, the anti-LAG-3 antibody is REGN3767.

In some aspects, the anti-LAG-3 antibody is LAG525.

In some aspects, the anti-CTLA-4 antibody is a bispecific antibody.

In some aspects, the anti-LAG-3 antibody is a bispecific antibody.

In some aspects, a bispecific antibody binds both CTLA-4 and LAG-3. In some aspects, a CTLA-4/LAG-3 bispecific antibody is XmAb22841.

In some aspects, a bispecific antibody binds both PD-1 and LAG-3. In some aspects, the PD-1/LAG-3 bispecific antibody is TSR-075. In some aspects, the PD-1/LAG-3 bispecific antibody is MGD013.

In some aspects, an antibody is a PD-L1/LAG-3 bispecific antibody. In some aspects, the PD-L1/LAG-3 bispecific antibody is FS-118.

In some aspects, in any of the methods of the disclosure, the patient is not additionally administered an anti-PD-1 antibody.

### VII. Outcomes

A patient treated according to the methods disclosed herein preferably experience improvement in at least one sign of melanoma. In one aspect, improvement is measured by a reduction in the quantity and/or size of measurable tumor lesions. In another aspect, lesions can be measured on chest x-rays or CT or MRI films. In another aspect, cytology or histology can be used to evaluate responsiveness to a therapy.

In one aspect, the patient treated exhibits a complete response (CR), a partial response (PR), stable disease (SD), immune-related complete disease (irCR), immune-related partial response (irPR), or immune-related stable disease (irSD). In another aspect, the patient treated experiences tumor shrinkage and/or decrease in growth rate, i.e., suppression of tumor growth. In another aspect, unwanted cell proliferation is reduced or inhibited. In yet another aspect, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other aspects, administration of effective amounts of the anti-LAG-3 antibody or the anti-LAG-3 antibody and anti-CTLA-4 antibody according to any of the methods provided herein produces at least one therapeutic effect selected from the group consisting of reduction in size of a tumor, reduction in number of metastatic lesions appearing over time, complete remission, partial remission, or stable disease.

In still other aspects, the methods of treatment produce a clinical benefit rate (CBR=CR+PR+SD≥6 months) better than that achieved by a method of treatment that does not comprise a step of (i) determining the level of LAG-3, PD-L1 and/or BRAF V600 expression in a tumor sample prior to treatment, and (ii) treating the tumor. In other aspects, the improvement of clinical benefit rate is about 20% 20%, 30%, 40%, 50%, 60%, 70%, 80% or more compared to a method of treatment that does not comprise a step of (i) determining the level of LAG-3, PD-L1, and/or BRAF V600 expression in a tumor sample prior to treatment, and (ii) treating the tumor. In still other aspects, the methods of treatment produce an objective response rate (ORR=CR+PR) of at least about 15%, at least about 20%, at least about 25%, at least about 30%, at least about 40%, at least about 50%, at least about 60%, at least about 70%, at least about 80%, at least about 90%, or about 100%.

### VIII. Kits and Unit Dosage Forms

Also within the scope of the present invention are diagnostic kits comprising an anti-LAG-3 antibody for assaying LAG-3 expression as a biomarker for screening patients for the immunotherapy or for predicting the efficacy of the immunotherapy. Kits typically include a label indicating the intended use of the contents of the kit and instructions for use. The term “label” includes any writing, or recorded material supplied on or with the kit, or which otherwise accompanies the kit. In certain aspects of a diagnostic kit, a first anti-LAG-3 antibody for assaying, detecting, and/or quantifying LAG-3 expression is co-packaged with at least one therapeutic antibody (e.g., a second anti-LAG-3 antibody or a second anti-LAG-3 antibody and an anti-CTLA-4 antibody) for the treatment of unresectable or metastatic melanoma. In some aspects, the kit further comprises an anti-PD-L1 antibody for assaying, detecting, and/or quantifying PD-L1 expression as a biomarker for predicting the efficacy of the immunotherapy. In one aspect, the immunotherapy comprises administering to the patient a therapeutically effective amount of a LAG-3 antagonist (e.g., anti-LAG-3 antibody) and a CTLA-4 inhibitor (e.g., an anti-CTLA-4 antibody).

In certain aspects, the diagnostic kit comprises an anti-human LAG-3 monoclonal antibody for assaying, detecting, and/or quantifying LAG-3 expression. See, e.g., J. Matsuzaki, et al.; PNAS 107, 7875 (2010).

Also provided herein are therapeutic kits which include a pharmaceutical composition containing an anti-LAG-3 antibody, such as BMS-986016, or an anti-LAG-3 antibody, such as BMS-986016 and an anti-CTLA-4 antibody, such as ipilimumab, in a therapeutically effective amount adapted for use in any of the methods of the disclosure. In certain aspects of a therapeutic kit, the anti-LAG-3 antibody is co-packaged with an anti-CTLA-4 antibody in unit dosage form. The kits optionally also can include instructions, e.g., comprising administration schedules, to allow a practitioner (e.g., a physician, nurse, or patient) to administer the composition contained therein to administer the composition to a patient having cancer (e.g., a solid tumor). The kit also can include a syringe.

Optionally, the diagnostic and/or therapeutic kits include multiple packages of the single-dose pharmaceutical compositions each containing an effective amount of the anti-LAG-3 or anti-CTLA-4 antibody for a single administration in accordance with any of the methods of the disclosure. Instruments or devices necessary for administering the pharmaceutical composition(s) also may be included in the kits. For instance, a kit may provide one or more pre-filled syringes containing an amount of the anti-LAG-3 or anti-CTLA-4 antibody.

In one aspect, the present invention provides a kit for treating a patient afflicted with unresectable or metastatic melanoma, the kit, for example, comprising: (a) a dose of an anti-LAG-3 antibody, such as one comprising CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5; (b) a dose of an anti-CTLA-4 antibody, such as one comprising CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32; and (c) instructions for using the anti-LAG-3 antibody and anti-CTLA-4 antibody in any of the methods described herein.

The present invention is further illustrated by the following examples which should not be construed as further limiting. The contents of all references cited throughout this application are expressly incorporated herein by reference.

### EXAMPLES

### Example 1

Efficacy of Anti-Lymphocyte Activation Gene-3 Antibody (Anti-LAG-3: BMS-986016) in Combination with Ipilimumab in Patients with Metastatic or Unresectable Melanoma

The purpose of this study is to evaluate the combination of BMS-986016 (relatlimab) and ipilimumab in the treatment of metastatic or unresectable melanoma.

Patients are selected based on the following inclusion criteria: (1) documented progression while on a prior anti-programmed cell death protein 1 (PD-1) containing regimen limited to Nivolumab or Pembrolizumab; (2) women of childbearing potential (WOCBP) must have a negative serum or urine pregnancy test; (3) participants must have histologically confirmed advanced unresectable (Stage III) or metastatic (Stage IV) melanoma, as per AJCC staging system; (4) tumor tissue from an unresectable or metastatic site of disease must be provided for biomarker analyses; (5) tumor tissue from an unresectable or metastatic site of disease must be provided for biomarker analyses; (6) Eastern Cooperative Oncology Group (ECOG) 0-1; and (7) ability to comply with treatment, patient-reported outcomes (PROs), PK, and pharmacodynamic sample collection and required study follow-up. Patients are selected based on the following exclusion criteria: (1) history of uveal melanoma; (2) known history of positive test for human immunodeficiency virus (HIV) or known acquired immunodeficiency syndrome; (3) prior treatment with ipilimumab, relatlimab, or any other CTLA-4 or LAG-3 targeted agents; and (4) positive blood screen for hepatitis C antibody, hepatitis B surface antigen, or HIV-1 and HIV-2 antibody.

During the treatment phase, patients will receive BMS-986016 (relatlimab) at a dose of 360 mg, 720 mg, 1080 mg or 1200 mg and ipilimumab at a dose of 3 mg/kg for each treatment cycle every three weeks.

SEQUENCES

SEQ ID NO: 1 Heavy Chain Amino Acid Sequence;
Anti-LAG-3 mAb (BMS-986016)
QVQLQQWGAGLLKPSETLSLTCAVYGGSFSDYYWN
WIRQPPGKGLEWIGEINHRGSTNSNPSLKSRVTLS
LDTSKNQFSLKLRSVTAADTAVYYCAFGYSDYEYN
WFDPWGQGTLVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK
SEQ ID NO: 2 Light Chain Amino Acid Sequence;
Anti-LAG-3 mAb (BMS-986016)
EIVLTQSPATLSLSPGERATLSCRASQSISSYLAW
YQQKPGQAPRLLIYDASNRATGIPARFSGSGSGTD
FTLTISSLEPEDFAVYYCQQRSNWPLTFGQGTNLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC
SEQ ID NO: 3 Heavy Chain Variable Region (VH)
Amino Acid Sequence; Anti-LAG-3 mAb
(BMS-986016)
QVQLQQWGAGLLKPSETLSLTCAVYGGSFSDYYWN
WIRQPPGKGLEWIGEINHRGSTNSNPSLKSRVTLS
LDTSKNQFSLKLRSVTAADTAVYYCAFGYSDYEYN
WFDPWGQGTLVTVSS
SEQ ID NO: 4 Heavy Chain Variable Region (VH)
Nucleotide Sequence; Anti-LAG-3 mAb
(BMS-986016)
caggtgcagctacagcagtggggcgcaggactgtt
gaagccttcggagaccctgtccctcacctgcgctg
tctatggtgggtccttcagtgattactactggaac
tggatccgccagcccccagggaaggggctggagtg
gattggggaaatcaatcatcgtggaagcaccaact
ccaacccgtccctcaagagtcgagtcaccctatca
ctagacacgtccaagaaccagttctccctgaagct
gaggtctgtgaccgccgcggacacggctgtgtatt
actgtgcgtttggatatagtgactacgagtacaac
tggttcgacccctggggccagggaaccctggtcac
cgtctcctca
SEQ ID NO: 5 Light Chain Variable Region (VL)
Amino Acid Sequence; Anti-LAG-3 mAb
(BMS-986016)
EIVLTQSPATLSLSPGERATLSCRASQSISSYLAW
YQQKPGQAPRLLIYDASNRATGIPARFSGSGSGTD
FTLTISSLEPEDFAVYYCQQRSNWPLTFGQGTNLE
IK
SEQ ID NO: 6 Light Chain Variable Region (VL)
Nucleotide Sequence; Anti-LAG-3 mAb
(BMS-986016)
gaaattgtgttgacacagtctccagccaccctgtc
tttgtctccaggggaaagagccaccctctcctgca
gggccagtcagagtattagcagctacttagcctgg
taccaacagaaacctggccaggctcccaggctcct
catctatgatgcatccaacagggccactggcatcc
cagccaggttcagtggcagtgggtctgggacagac
ttcactctcaccatcagcagcctagagcctgaaga
ttttgcagtttattactgtcagcagcgtagcaact
ggcctctcacttttggccaggggaccaacctggag
atcaaa
SEQ ID NO: 7 Heavy Chain CDR1 Amino Acid
Sequence; Anti-LAG-3 mAb (BMS-986016)
DYYWN
SEQ ID NO: 8 Heavy Chain CDR2 Amino Acid
Sequence; Anti-LAG-3 mAb (BMS-986016)
EINHRGSTNSNPSLKS
SEQ ID NO: 9 Heavy Chain CDR3 Amino Acid
Sequence; Anti-LAG-3 mAb (BMS-986016)
GYSDYEYNWFDP
SEQ ID NO: 10 Light Chain CDR1 Amino Acid
Sequence; Anti-LAG-3 mAb (BMS-986016)
RASQSISSYLA
SEQ ID NO: 11 Light Chain CDR2 Amino Acid
Sequence; Anti-LAG-3 mAb (BMS-986016)
DASNRAT
SEQ ID NO: 12 Light Chain CDR3 Amino Acid
Sequence; Anti-LAG-3 mAb (BMS-986016)
QQRSNWPLT
SEQ ID NO: 13 Heavy Chain Amino Acid Sequence;
Anti-PD-1 mAb (BMS936558)
QVQLVESGGGVVQPGRSLRLDCKASGITFSNSGMH
WVRQAPGKGLEWVAVIWYDGSKRYYADSVKGRFTI
SRDNSKNTLFLQMNSLRAEDTAVYYCATNDDYWGQ
GTLVTVSSASTKGPSVFPLAPCSRSTSESTAALGC
LVKDYFPEPVTVSWNSGALTSGVHTFPAVLQSSGL
YSLSSVVTVPSSSLGTKTYTCNVDHKPSNTKVDKR
VESKYGPPCPPCPAPEFLGGPSVFLFPPKPKDTLM
ISRTPEVTCVVVDVSQEDPEVQFNWYVDGVEVHNA
KTKPREEQFNSTYRVVSVLTVLHQDWLNGKEYKCK
VSNKGLPSSIEKTISKAKGQPREPQVYTLPPSQEE
MTKNQVSLTCLVKGFYPSDIAVEWESNGQPENNYK
TTPPVLDSDGSFFLYSRLTVDKSRWQEGNVFSCSV
MHEALHNHYTQKSLSLSLGK
SEQ ID NO: 14 Light Chain Amino Acid Sequence;
Anti-PD-1 mAb (BMS936558)
EIVLTQSPATLSLSPGERATLSCRASQSVSSYLAW
YQQKPGQAPRLLIYDASNRATGIPARFSGSGSGTD
FTLTISSLEPEDFAVYYCQQSSNWPRTFGQGTKVE
IKRTVAAPSVFIFPPSDEQLKSGTASVVCLLNNFY
PREAKVQWKVDNALQSGNSQESVTEQDSKDSTYSL
SSTLLSKADYEKHKVYACEVTHQGLSSPVTKSFNR
GEC
SEQ ID NO: 15 Heavy Chain Variable Region (VH)
Amino Acid Sequence; Anti-PD-1 mAb
(BMS936558)
QVQLVESGGGVVQPGRSLRLDCKASGITFSNSGMH
WVRQAPGKGLEWVAVIWYDGSKRYYADSVKGRFTI
SRDNSKNTLFLQMNSLRAEDTAVYYCATNDDYWGQ
GTLVTVSS
SEQ ID NO: 16 Heavy Chain Variable Region (VH)
Nucleotide Sequence; Anti-PD-1 mAb
(BMS936558)
caggtgcagctggtggagtctgggggaggcgtggt
ccagcctgggaggtccctgagactcgactgtaaag
cgtctggaatcaccttcagtaactctggcatgcac
tgggtccgccaggctccaggcaaggggctggagtg
ggtggcagttatttggtatgatggaagtaaaagat
actatgcagactccgtgaagggccgattcaccatc
tccagagacaattccaagaacacgctgtttctgca
aatgaacagcctgagagccgaggacacggctgtgt
attactgtgcgacaaacgacgactactggggccag
ggaaccctggtcaccgtctcctca
SEQ ID NO: 17 Light Chain Variable Region (VL)
Amino Acid Sequence; Anti-PD-1 mAb
(BMS936558)
EIVLTQSPATLSLSPGERATLSCRASQSVSSYLAW
YQQKPGQAPRLLIYDASNRATGIPARFSGSGSGTD
FTLTISSLEPEDFAVYYCQQSSNWPRTFGQGTKVE
IK
SEQ ID NO: 18 Light Chain Variable Region (VL)
Nucleotide Sequence; Anti-PD-1 mAb
(BMS936558)
gaaattgtgttgacacagtctccagccaccctgtc
tttgtctccaggggaaagagccaccctctcctgca
gggccagtcagagtgttagtagttacttagcctgg
taccaacagaaacctggccaggctcccaggctcct
catctatgatgcatccaacagggccactggcatcc
cagccaggttcagtggcagtgggtctgggacagac
ttcactctcaccatcagcagcctagagcctgaaga
ttttgcagtttattactgtcagcagagtagcaact
ggcctcggacgttcggccaagggaccaaggtggaa
atcaaa
SEQ ID NO: 19 Heavy Chain CDR1 Amino Acid
Sequence; Anti-PD-1 mAb (BMS936558)
NSGMH
SEQ ID NO: 20 Heavy Chain CDR2 Amino Acid
Sequence; Anti-PD-1 mAb (BMS936558)
VIWYDGSKRYYADSVKG
SEQ ID NO: 21 Heavy Chain CDR3 Amino Acid
Sequence; Anti-PD-1 mAb (BMS936558)
NDDY
SEQ ID NO: 22 Light Chain CDR1 Amino Acid
Sequence; Anti-PD-1 mAb (BMS936558)
RASQSVSSYLA
SEQ ID NO: 23 Light Chain CDR2 Amino Acid
Sequence; Anti-PD-1 mAb (BMS936558)
DASNRAT
SEQ ID NO: 24 Light Chain CDR3 Amino Acid
Sequence; Anti-PD-1 mAb (BMS936558)
QQSSNWPRT
SEQ ID NO: 25 Heavy Chain Nucleotide Sequence;
Anti-LAG-3 mAb (BMS-986016)
caggtgcagctacagcagtggggcgcaggactgtt
gaagccttcggagaccctgtccctcacctgcgctg
tctatggtgggtccttcagtgattactactggaac
tggatccgccagcccccagggaaggggctggagtg
gattggggaaatcaatcatcgtggaagcaccaact
ccaacccgtccctcaagagtcgagtcaccctatca
ctagacacgtccaagaaccagttctccctgaagct
gaggtctgtgaccgccgcggacacggctgtgtatt
actgtgcgtttggatatagtgactacgagtacaac
tggttcgacccctggggccagggaaccctggtcac
cgtctcctcagctagcaccaagggcccatccgtct
tccccctggcgccctgctccaggagcacctccgag
agcacagccgccctgggctgcctggtcaaggacta
cttccccgaaccggtgacggtgtcgtggaactcag
gcgccctgaccagcggcgtgcacaccttcccggct
gtcctacagtcctcaggactctactccctcagcag
cgtggtgaccgtgccctccagcagcttgggcacga
agacctacacctgcaacgtagatcacaagcccagc
aacaccaaggtggacaagagagttgagtccaaata
tggtcccccatgcccaccatgcccagcacctgagt
tcctggggggaccatcagtcttcctgttcccccca
aaacccaaggacactctcatgatctcccggacccc
tgaggtcacgtgcgtggtggtggacgtgagccagg
aagaccccgaggtccagttcaactggtacgtggat
ggcgtggaggtgcataatgccaagacaaagccgcg
ggaggagcagttcaacagcacgtaccgtgtggtca
gcgtcctcaccgtcctgcaccaggactggctgaac
ggcaaggagtacaagtgcaaggtctccaacaaagg
cctcccgtcctccatcgagaaaaccatctccaaag
ccaaagggcagccccgagagccacaggtgtacacc
ctgcccccatcccaggaggagatgaccaagaacca
ggtcagcctgacctgcctggtcaaaggcttctacc
ccagcgacatcgccgtggagtgggagagcaatggg
cagccggagaacaactacaagaccacgcctcccgt
gctggactccgacggctccttcttcctctacagca
ggctaaccgtggacaagagcaggtggcaggagggg
aatgtcttctcatgctccgtgatgcatgaggctct
gcacaaccactacacacagaagagcctctccctgt
ctctgggtaaatga
SEQ ID NO: 26 Light Chain Nucleotide Sequence;
Anti-LAG-3 mAb (BMS-986016)
gaaattgtgttgacacagtctccagccaccctgtc
tttgtctccaggggaaagagccaccctctcctgca
gggccagtcagagtattagcagctacttagcctgg
taccaacagaaacctggccaggctcccaggctcct
catctatgatgcatccaacagggccactggcatcc
cagccaggttcagtggcagtgggtctgggacagac
ttcactctcaccatcagcagcctagagcctgaaga
ttttgcagtttattactgtcagcagcgtagcaact
ggcctctcacttttggccaggggaccaacctggag
atcaaacgtacggtggctgcaccatctgtcttcat
cttcccgccatctgatgagcagttgaaatctggaa
ctgcctctgttgtgtgcctgctgaataacttctat
cccagagaggccaaagtacagtggaaggtggataa
cgccctccaatcgggtaactcccaggagagtgtca
cagagcaggacagcaaggacagcacctacagcctc
agcagcaccctgacgctgagcaaagcagactacga
gaaacacaaagtctacgcctgcgaagtcacccatc
agggcctgagctcgcccgtcacaaagagcttcaac
aggggagagtgttag
SEQ ID NO: 27 LAG-3 epitope
PGHPLAPG
SEQ ID NO: 28 LAG-3 epitope
HPAAPSSW
SEQ ID NO: 29 LAG-3 epitope
PAAPSSWG
SEQ ID NO: 30 Heavy Chain Amino Acid Sequence;
Anti-LAG-3 mAb (BMS-986016) without
terminal lysine
QVQLQQWGAGLLKPSETLSLTCAVYGGSFSDYYWN
WIRQPPGKGLEWIGEINHRGSTNSNPSLKSRVTLS
LDTSKNQFSLKLRSVTAADTAVYYCAFGYSDYEYN
WFDPWGQGTLVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLG
SEQ ID NO: 31 light chain variable region
(Vk), 10D1 from Vk A-27
gaaattgtgttgacgcagtctccaggcaccctgtc
tttgtctccaggggaaagagccaccctctcctgca
gggccagtcagagtgttggcagcagctacttagcc
tggtaccagcagaaacctggccaggctcccaggct
cctcatctatggtgcattcagcagggccactggca
tcccagacaggttcagtggcagtgggtctgggaca
gacttcactctcaccatcagcagactggagcctga
agattttgcagtgtattactgtcagcagtatggta
gctcaccgtggacgttcggccaagggaccaaggtg
gaaatcaaac
SEQ ID NO: 32 light chain variable region
predicted sequence for 10D1
EIVLTQSPGTLSLSPGERATLSCRASQSVGSSYLA
WYQQKPGQAPRLLIYGAFSRATGIPDRFSGSGSGT
DFTLTISRLEPEDFAVYYCQQYGSSPWTFGQGTKV
EIK
SEQ ID NO: 33 heavy chain variable region 10D1
caggtgcagctggtggagtctgggggaggcgtggt
ccagcctgggaggtccctgagactctcctgtgcag
cctctggattcaccttcagtagctatactatgcac
tgggtccgccaggctccaggcaaggggctggagtg
ggtgacatttatatcatatgatggaaacaataaat
actacgcagactccgtgaagggccgattcaccatc
tccagagacaattccaagaacacgctgtatctgca
aatgaacagcctgagagctgaggacacggctatat
attactgtgcgaggaccggctggctggggcccttt
gactactggggccagggaaccctggtcaccgtctc
ctcag
SEQ ID NO: 34 heavy chain variable region
predicted sequence for 10D1 from VH 3-30.3
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSS
SEQ ID NO: 35 10D1 HC CDR1
SYTMH
SEQ ID NO: 36 10D1 HC CDR2
FISYDGNNKYYADSVKG
SEQ ID NO: 37 10D1 HC CDR3
TGWLGPFDY
SEQ ID NO: 38 10D1 LC CDR1
RASQSVGSSYLA
SEQ ID NO: 39 10D1 LC CDR2
GAFSRAT
SEQ ID NO: 40 10D1 LC CDR3
QQYGSSPWT
SEQ ID NO: 41 Lymphocyte Activation Gene 3
Protein Amino Acid Sequence (Homo Sapiens,
NP_002277)
MWEAQFLGLLFLQPLWVAPVKPLQPGAEVPVVWAQ
EGAPAQLPCSPTIPLQDLSLLRRAGVTWQHQPDSG
PPAAAPGHPLAPGPHPAAPSSWGPRPRRYTVLSVG
PGGLRSGRLPLQPRVQLDERGRQRGDFSLWLRPAR
RADAGEYRAAVHLRDRALSCRLRLRLGQASMTASP
PGSLRASDWVILNCSFSRPDRPASVHWFRNRGQGR
VPVRESPHHHLAESFLFLPQVSPMDSGPWGCILTY
RDGFNVSIMYNLTVLGLEPPTPLTVYAGAGSRVGL
PCRLPAGVGTRSFLTAKWTPPGGGPDLLVTGDNGD
FTLRLEDVSQAQAGTYTCHIHLQEQQLNATVTLAI
ITVTPKSFGSPGSLGKLLCEVTPVSGQERFVWSSL
DTPSQRSFSGPWLEAQEAQLLSQPWQCQLYQGERL
LGAAVYFTELSSPGAQRSGRAPGALPAGHLLLFLI
LGVLSLLLLVTGAFGFHLWRRQWRPRRFSALEQGI
HPPQAQSKIEELEQEPEPEPEPEPEPEPEPEPEQL

1-175. (canceled)

176. A method of treating an unresectable or metastatic melanoma in a human patient, the method comprising administering to the patient:
(a) a lymphocyte activation gene-3 (LAG-3) antagonist, and
(b) a cytotoxic T-lymphocyte antigen-4 (CTLA-4) inhibitor;
wherein the patient has a sensitizing mutation for a targeted inhibitor therapy and/or has received a prior programmed death-1 (PD-1) pathway inhibitor as a treatment for melanoma.

177. The method of claim 176, wherein the patient has a B-rapidly accelerated fibrosarcoma proto-oncogene (BRAF), mitogen-activated extracellular signal-regulated kinase kinase (MEK), neuroblastoma RAS viral oncogene homolog (NRAS), and/or proto-oncogene c-KIT (KIT) mutation sensitive to targeted inhibitor therapy.

178. The method of claim 176, wherein the patient has histologically confirmed unresectable stage III or stage IV melanoma.

179. The method of claim 176, wherein at least about 1% of immune cells in tumor tissue from the patient express LAG-3 and/or at least about 1% of tumor cells in tumor tissue from the patient express PD-L1.

180. The method of claim 176, wherein the LAG-3 antagonist comprises a soluble LAG-3 polypeptide or an anti-LAG-3 antibody.

181. The method of claim 180, wherein the soluble LAG-3 polypeptide comprises IMP321 (eftilagimod alpha), or wherein the anti-LAG-3 antibody comprises:
(a) a full-length antibody,
(b) a monoclonal, human, humanized, chimeric, or multispecific antibody,
(c) a dual-affinity re-targeting antibody (DART), a DVD-Ig, or a bispecific antibody,
(d) a F(ab′)2 fragment, a Fab′ fragment, a Fab fragment, a Fv fragment, a scFv fragment, a dsFv fragment, a dAb fragment, or a single chain binding polypeptide,
(e) BMS-986016 (relatlimab), IMP731 (H5L7BW), MK4280 (28G-10), REGN3767 (fianlimab), GSK2831781, humanized BAP050, IMP-701 (LAG525, ieramilimab), aLAG3(0414), aLAG3(0416), Sym022, TSR-033, TSR-075, XmAb22841, MGD013, B1754111, FS118, P 13B02-30, AVA-017, 25F7, AGEN1746, or an antigen binding portion thereof,
(f) CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5,
(g) a heavy chain variable region CDR1, CDR, and CDR3 comprising the sequences set forth in SEQ ID NOs:7, 8, and 9, respectively, and a light chain variable region CDR1, CDR, and CDR3 comprising the sequences set forth in SEQ ID NOs:10, 11, and 12, respectively,
(h) heavy and light chain variable regions comprising the sequences set forth in SEQ ID NOs:3 and 5, respectively,
(i) heavy and light chains comprising the sequences set forth in SEQ ID NOs:1 and 2, respectively, or
(j) heavy and light chains comprising the sequences set forth in SEQ ID NOs:30 and 2, respectively.

182. The method of claim 176, wherein the CTLA-4 inhibitor comprises an anti-CTLA-4 antibody.

183. The method of claim 182, wherein the anti-CTLA-4 antibody comprises:
(a) a full-length antibody,
(b) a monoclonal, human, humanized, chimeric, or multispecific antibody,
(c) a DART, a DVD-Ig, or a bispecific antibody,
(d) a F(ab′)2 fragment, a Fab′ fragment, a Fab fragment, a Fv fragment, a scFv fragment, a dsFv fragment, a dAb fragment, or a single chain binding polypeptide,
(e) ipilimumab, tremelimumab, MK-1308, AGEN-1884, or an antigen binding portion thereof,
(f) CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32,
(g) a heavy chain variable region CDR1, CDR, and CDR3 comprising the sequences set forth in SEQ ID NOs:35, 36, and 37, respectively, and a light chain variable region CDR1, CDR, and CDR3 comprising the sequences set forth in SEQ ID NOs:38, 39, and 40, respectively,
(h) heavy and light chain variable regions comprising the sequences set forth in SEQ ID NOs:34 and 32, respectively, or
(i) the heavy and light chains of ipilimumab.

184. The method of claim 176, wherein the LAG-3 antagonist and the CTLA-4 inhibitor are formulated for parenteral administration.

185. The method of claim 184, wherein the LAG-3 antagonist and the CTLA-4 inhibitor are formulated for intravenous, intramuscular, subcutaneous, intraperitoneal, or spinal administration.

186. The method of claim 176, wherein the LAG-3 antagonist and the CTLA-4 inhibitor are formulated separately.

187. The method of claim 176, wherein the LAG-3 antagonist and the CTLA-4 inhibitor are formulated together.

188. The method of claim 176, further comprising administering to the patient a tyrosine kinase inhibitor, an anti-angiogenesis agent, a checkpoint inhibitor, a checkpoint stimulator, a chemotherapeutic agent, an immunotherapeutic agent, a platinum agent, an alkylating agent, a taxane, a nucleoside analog, an antimetabolite, a topisomerase inhibitor, an anthracycline, a vinca alkaloid, or any combination thereof.

189. The method of claim 188, wherein the checkpoint inhibitor comprises a PD-1 pathway inhibitor, a T cell immunoglobulin and ITIM domain (TIGIT) inhibitor, a T cell immunoglobulin and mucin-domain containing-3 (TIM-3) inhibitor, a TIM-1 inhibitor, a TIM-4 inhibitor, a B7-H3 inhibitor, a B7-H4 inhibitor, a B and T cell lymphocyte attenuator (BTLA) inhibitor, a V-domain Ig suppressor of T cell activation (VISTA) inhibitor, an indoleamine 2,3-dioxygenase (IDO) inhibitor, a nicotinamide adenine dinucleotide phosphate oxidase isoform 2 (NOX2) inhibitor, a killer-cell immunoglobulin-like receptor (KIR) inhibitor, an adenosine A2a receptor (A2aR) inhibitor, a transforming growth factor beta (TGF-β) inhibitor, a phosphoinositide 3-kinase (PI3K) inhibitor, a CD47 inhibitor, a CD48 inhibitor, a CD73 inhibitor, a CD113 inhibitor, a sialic acid-binding immunoglobulin-like lectin-7 (SIGLEC-7) inhibitor, a SIGLEC-9 inhibitor, a SIGLEC-15 inhibitor, a glucocorticoid-induced TNFR-related protein (GITR) inhibitor, a galectin-1 inhibitor, a galectin-9 inhibitor, a carcinoembryonic antigen-related cell adhesion molecule-1 (CEACAM-1) inhibitor, a G protein-coupled receptor 56 (GPR56) inhibitor, a glycoprotein A repetitions predominant (GARP) inhibitor, a 2B4 inhibitor, a programmed death-1 homolog (PD1H) inhibitor, a leukocyte-associated immunoglobulin-like receptor 1 (LAIR1) inhibitor, or any combination thereof.

190. The method of claim 189, wherein the PD-1 pathway inhibitor comprises a soluble PD-L2 polypeptide, an anti-PD-1 antibody and/or an anti-PD-L1 antibody, or BMS-986189.

191. The method of claim 190, wherein the soluble PD-L2 polypeptide comprises AMP-224, and/or wherein:
(a) the anti-PD-1 antibody and/or the anti-PD-L1 antibody comprises a full-length antibody,
(b) the anti-PD-1 antibody and/or the anti-PD-L1 antibody comprises a monoclonal, human, humanized, chimeric, or multispecific antibody,
(c) the anti-PD-1 antibody and/or the anti-PD-L1 antibody comprises a DART, a DVD-Ig, or a bispecific antibody,
(d) the anti-PD-1 antibody and/or the anti-PD-L1 antibody comprises a F(ab′)2 fragment, a Fab′ fragment, a Fab fragment, a Fv fragment, a scFv fragment, a dsFv fragment, a dAb fragment, or a single chain binding polypeptide, or
(e) the anti-PD-1 antibody comprises: nivolumab, pembrolizumab, PDR001 (spartalizumab), MEDI-0680, TSR-042, cemiplimab, JS001, PF-06801591, BGB-A317, BI 754091, INCSHR1210, GLS-010, AM-001, STI-1110, AGEN2034, MGA012, BCD-100, IBI308, SSI-361, or an antigen binding portion thereof, and/or the anti-PD-L1 antibody comprises: BMS-936559, atezolizumab, durvalumab, avelumab, STI-1014, CX-072, KN035, LY3300054, BGB-A333, ICO 36, CK-301, or an antigen binding portion thereof.

192. A method of treating an unresectable or metastatic melanoma in a human patient, the method comprising administering to the patient:
(a) a dose of about 360 mg, about 720 mg, about 1080 mg, or about 1200 mg of an anti-LAG-3 antibody, and
(b) a dose of about 3 mg/kg of an anti-CTLA-4 antibody.

193. The method of claim 192, wherein the patient has received a prior anti-PD-1 antibody as a treatment for melanoma.

194. The method of claim 192, wherein:
(a) the anti-LAG-3 antibody and/or the anti-CTLA-4 antibody comprises a full-length antibody,
(b) the anti-LAG-3 antibody and/or the anti-CTLA-4 antibody comprises a monoclonal, human, humanized, chimeric, or multispecific antibody,
(c) the anti-LAG-3 antibody and/or the anti-CTLA-4 antibody comprises a DART, a DVD-Ig, or a bispecific antibody,
(d) the anti-LAG-3 antibody and/or the anti-CTLA-4 antibody comprises a F(ab′)2 fragment, a Fab′ fragment, a Fab fragment, a Fv fragment, a scFv fragment, a dsFv fragment, a dAb fragment, or a single chain binding polypeptide, or
(e) (i) the anti-LAG-3 antibody comprises:
(1) BMS-986016 (relatlimab), IMP731 (H5L7BW), MK4280 (28G-10), REGN3767 (fianlimab), GSK2831781, humanized BAP050, IMP-701 (LAG525, ieramilimab), aLAG3(0414), aLAG3(0416), Sym022, TSR-033, TSR-075, XmAb22841, MGD013, BI754111, FS118, P 13B02-30, AVA-017, 25F7, AGEN1746, or an antigen binding portion thereof,
(2) CDR1, CDR2 and CDR3 domains of the heavy chain variable region having the sequence set forth in SEQ ID NO:3, and CDR1, CDR2 and CDR3 domains of the light chain variable region having the sequence set forth in SEQ ID NO:5,
(3) a heavy chain variable region CDR1, CDR, and CDR3 comprising the sequences set forth in SEQ ID NOs:7, 8, and 9, respectively, and a light chain variable region CDR1, CDR, and CDR3 comprising the sequences set forth in SEQ ID NOs:10, 11, and 12, respectively,
(4) heavy and light chain variable regions comprising the sequences set forth in SEQ ID NOs:3 and 5, respectively,
(5) heavy and light chains comprising the sequences set forth in SEQ ID NOs:1 and 2, respectively, or
(6) heavy and light chains comprising the sequences set forth in SEQ ID NOs:30 and 2, respectively, and
(ii) the anti-CTLA-4 antibody comprises:
(1) ipilimumab, tremelimumab, MK-1308, AGEN-1884, or an antigen binding portion thereof,
(2) CDR1, CDR2 and CDR3 domains of the heavy chain variable region having the sequence set forth in SEQ ID NO:34, and CDR1, CDR2 and CDR3 domains of the light chain variable region having the sequence set forth in SEQ ID NO:32,
(3) a heavy chain variable region CDR1, CDR, and CDR3 comprising the sequences set forth in SEQ ID NOs:35, 36, and 37, respectively, and a light chain variable region CDR1, CDR, and CDR3 comprising the sequences set forth in SEQ ID NOs:38, 39, and 40, respectively,
(4) heavy and light chain variable regions comprising the sequences set forth in SEQ ID NOs:34 and 32, respectively, or
(5) the heavy and light chains of ipilimumab.

195. A method of treating an unresectable or metastatic melanoma in a human patient, the method comprising administering to the patient an anti-LAG-3 antibody comprising:
(a) a heavy chain variable region CDR1 comprising the sequence set forth in SEQ ID NO:7;
(b) a heavy chain variable region CDR2 comprising the sequence set forth in SEQ ID NO:8;
(c) a heavy chain variable region CDR3 comprising the sequence set forth in SEQ ID NO:9;
(d) a light chain variable region CDR1 comprising the sequence set forth in SEQ ID NO:10;
(e) a light chain variable region CDR2 comprising the sequence set forth in SEQ ID NO:11; and
(f) a light chain variable region CDR3 comprising the sequence set forth in SEQ ID NO:12, and
wherein the patient has received a prior anti-PD-1 antibody as a treatment for melanoma;
and wherein at least one dose of the anti-LAG-3 antibody is administered at a dose of about 360 mg, about 720 mg, about 1080 mg, or about 1200 mg.
